(19) United States
(12) Patent Application Publication
(10) Pub. No.: US 2024/0252557 A1
(43) Pub. Date: Aug. 1, 2024

(54) MULTIBIOTIC SUPPLEMENTS

(71) Applicant: Natals, Inc., Culver City, CA (US)

(72) Inventors: Nima ALAMDARI, Los Angeles, CA (US); Adeline M. GRIER-WELCH, Los Angeles, CA (US); Shilpa A. RAUT, Long Beach, CA (US); Mastaneh SHARAFI, Redondo Beach, CA (US); Katerina SCHNEIDER, Los Angeles, CA (US); Arianne Y. VANCE, Santa Monica, CA (US)

(21) Appl. No.: 18/424,585

(22) Filed: Jan. 26, 2024

Related U.S. Application Data
(60) Provisional application No. 63/481,990, filed on Jan. 27, 2023.

(51) Int. Cl. A61K 35/747; A61K 9/48; A61K 31/225; A61K 35/745; A61K 35/76; A61K 47/44; A61P 1/00

(52) U.S. Cl. CPC A61K 35/747; A61K 9/485; A61K 9/4858; A61K 9/4891; A61K 31/225; A61K 35/745; A61K 35/76; A61K 47/44; A61P 1/00

(57) ABSTRACT

Described herein are multi-biotic supplements containing a mixture of postbiotics, prebiotics, and probiotics within nested capsules, for use in supporting the health of a subject, including gut health, digestive health, and immune health.

### CROSS-REFERENCE TO RELATED APPLICATIONS

This application claims priority to U.S. Provisional Application No. 63/481,990 filed on Jan. 27, 2023, the contents of which are incorporated herein by reference in their entireties.

### TECHNICAL FIELD

The present disclosure relates generally to dietary supplements, and more specifically to multi-biotic dietary supplements that include a postbiotic or postbiotic precursor, a probiotic blend, and/or a prebiotic blend.

### BACKGROUND

The gut microbiome plays an important role in human health. For instance, microorganisms in the gut produce cytokines that protect from pathogens, metabolize otherwise indigestible compounds in food, and maintain the intestinal epithelium. Prebiotics, probiotics, and postbiotics are three types of supplements that are consumed to modulate the microbiome.

“Synbiotics” (supplements that include both probiotics and prebiotics) are commonly consumed to promote gut health. However, such synbiotics typically do not contain a large enough dose of prebiotics to be efficacious. Further, most synbiotics do not include any postbiotics, which often have an unpleasant taste and an offensive odor. The inclusion of postbiotics thus decreases user compliance.

There is a need, therefore, for a supplement that (i) contains effective doses of a probiotic, a postbiotic, and a prebiotic, (ii) enables the probiotic, postbiotic, and prebiotic to reach the locations in the gastrointestinal tract where they are each best able to aid the microbiome, and (iii) improves consumer compliance by masking the unpleasant taste and odor of the postbiotic and delivers all components in a single dose.

### SUMMARY

Supplements containing postbiotics, prebiotics, and probiotics are described herein. In some variations, these supplements have an inner capsules residing in an outer capsule. Such supplements may contain a probiotic and prebiotic, while the outer capsule may contain a postbiotic. Each of the inner and outer capsules may have different dissolution or degradation rates.

Also disclosed herein are methods for improving and/or supporting gut health, digestive health, and/or immune health in a subject by administered a supplement to a subject.

### BRIEF DESCRIPTION OF THE DRAWINGS

FIG. 1 shows a schematic representation of the Simulation of the Human Intestinal Microbial Ecosystem (SHIME) model used in Example 2.

FIG. 2 shows a schematic representation of the methods used for testing Supplement A in Example 2.

FIGS. 3A and 3B show the culturable population densities of probiotics obtained from the simulated passage of Supplement A through the upper gastrointestinal tract in Example 2.

FIG. 4 shows a schematic representation of the short-term colonic fermentation methods used for testing Supplement A in Example 3.

FIG. 5 shows the difference in butyrate production for Supplement A and three comparative examples, relative to a blank, after incubation for twenty-four hours.

FIG. 6 shows the effect of colonic batch suspensions on transepithelial electrical resistance (TEER) of the Caco-2/THP1-Blue™ co-cultures.

### DETAILED DESCRIPTION

The following description sets forth numerous exemplary configurations, methods, parameters, and the like. It should be recognized, however, that such description is not intended as a limitation on the scope of the present disclosure but is instead provided as a description of exemplary embodiments.

### Multi-Biotic Supplement Compositions

The present disclosure provides dietary supplements. In some embodiments, the dietary supplements may be multi-biotic supplements. In some embodiments, the multi-biotic supplements contain one or more of a postbiotic, a postbiotic precursor, a probiotic, and/or a prebiotic. In some embodiments, the multi-biotic supplements contain two or more of a postbiotic, a postbiotic precursor, a probiotic, and/or a prebiotic. In some embodiments, the multi-biotic supplements contain three or more of a postbiotic, a postbiotic precursor, a probiotic, and/or a prebiotic. The multi-biotic supplements of some embodiments contain a postbiotic or postbiotic precursor, a probiotic, and/or a prebiotic. The multi-biotic supplements of some embodiments contain each of a postbiotic, a probiotic, and/or a prebiotic. The multi-biotic supplements of some embodiments contain each of a postbiotic precursor, a probiotic, and/or a prebiotic.

### A. Postbiotics and Postbiotic Precursors

Generally, the multi-biotic supplements of this disclosure contain one or more of a postbiotic and/or a postbiotic precursor. In some embodiments, the multi-biotic supplement contains a postbiotic. Postbiotics are typically considered to be bioactive compounds that are produced by microorganisms during a fermentation process. For the purposes of this disclosure, it should be understood that a postbiotic or postbiotic precursor may be isolated from a microorganism or may be synthesized by any other means. In some embodiments, the postbiotic may be a carboxylic acid, carboxylate, or an ester. In some embodiments, the postbiotic may be a short-chain fatty acid. In some embodiments, the postbiotic may include one or more of acetic acid, propanoic acid, butanoic acid, valeric acid, caproic acid, and the conjugate bases thereof. In some embodiments, the postbiotic may include one or more selected from acetic acid, propanoic acid, butanoic acid, and the conjugate bases thereof. In some embodiments, the postbiotic may be an ester of a short-chain fatty acid. In some embodiments, the postbiotic may include one or more of an acetate, a propionate, a butyrate, a valerate and a caproate. In some embodiments, the postbiotic may include one or more selected from an acetate, a propionate, and a butyrate. In some embodiments, the postbiotic may include a butyrate. In some embodiments, the postbiotic is a butyric acid glycerol ester. In some embodiments, the postbiotic may comprise inactivated microbial cells.

In some embodiments, the multi-biotic supplement contains a postbiotic precursor. In some embodiments, the postbiotic precursor is a compound that can be metabolized to a postbiotic. In some embodiments, the postbiotic precursor is a compound that can be metabolized to a postbiotic in the gastrointestinal tract. In some embodiments, a metabolite of the postbiotic precursor may be a short-chain fatty acid. In some embodiments, a metabolite of the postbiotic precursor is one or more of acetic acid, propanoic acid, butanoic acid, valeric acid and caproic acid. In some embodiments, a metabolite of the postbiotic precursor is one or more selected from acetic acid, propanoic acid and butanoic acid. In some embodiments, a metabolite of the postbiotic precursor may be an ester of a short-chain fatty acid. In some embodiments, a metabolite of the postbiotic precursor is one or more of an acetate, a propionate, a butyrate, a valerate and a caproate. In some embodiments, a metabolite of the postbiotic precursor is one or more selected from an acetate, a propionate, and a butyrate. In some embodiments, a metabolite of the postbiotic precursor is a butyrate. In some embodiments, the postbiotic precursor is tributyrin. In some embodiments, the postbiotic precursor may comprise inactivated microbial cells.

In some embodiments, the multi-biotic supplement contains the postbiotic or postbiotic precursor in an amount of about 10 mg to about 1000 mg. In some embodiments, the multi-biotic supplement contains the postbiotic or postbiotic precursor in an amount of about 20 mg to about 900 mg. In some embodiments, the multi-biotic supplement contains the postbiotic or postbiotic precursor in an amount of about 30 mg to about 800 mg. In some embodiments, the multi-biotic supplement contains the postbiotic or postbiotic precursor in an amount of about 40 mg to about 700 mg. In some embodiments, the multi-biotic supplement contains the postbiotic or postbiotic precursor in an amount of about 50 mg to about 600 mg. In some embodiments, the multi-biotic supplement contains the postbiotic or postbiotic precursor in an amount of about 100 mg to about 500 mg. In some embodiments, the multi-biotic supplement contains the postbiotic or postbiotic precursor in an amount of about 150 mg to about 450 mg. In some embodiments, the multi-biotic supplement contains the postbiotic or postbiotic precursor in an amount of about 200 mg to about 400 mg. In some embodiments, the multi-biotic supplement contains the postbiotic or postbiotic precursor in an amount of about 250 mg to about 400 mg. In some embodiments, the multi-biotic supplement contains the postbiotic or postbiotic precursor in an amount of about 300 mg to about 400 mg. In some embodiments, the multi-biotic supplement contains the postbiotic or postbiotic precursor in an amount of about 250 mg to about 350 mg. In some embodiments, the multi-biotic supplement contains the postbiotic or postbiotic precursor in an amount selected from about 250 mg, about 275 mg, about 300 mg, about 325 mg, about 350 mg, about 375 mg, 400 mg, 425 mg, 450 mg, 475 mg, 500 mg, 525 mg, 550 mg, 575 mg, 600 mg, 625 mg, 650 mg, 675 mg, and 700 mg. In some embodiments, the multi-biotic supplement contains the postbiotic in an amount of about 350 mg. In some embodiments, the multi-biotic supplement contains the postbiotic precursor in an amount of about 350 mg. In some embodiments, the multi-biotic supplement contains the postbiotic in an amount of about 300 mg. In some embodiments, the multi-biotic supplement contains the postbiotic precursor in an amount of about 300 mg.

In some embodiments, the multi-biotic supplement contains the postbiotic or postbiotic precursor in a liquid form. In some embodiments, the multi-biotic supplement contains the postbiotic or postbiotic precursor in a solution. In some embodiments, the multi-biotic supplement contains the postbiotic or postbiotic precursor in a solution that includes one or more oils. In some embodiments, the oil is liquid above about −110° F., above about −100° F., above about −90° F., above about −80° F., above about −70° F., above about −60° F., above about −50° F., −40° F., above about −30° F., above about −20° F., above, about −10° F., above about 0° F., above about 10° F., above about 20° F., above about 30° F., above about 40° F., above about 50° F., above about 60° F., above about 70° F., above about 80° F., or above about 90° F. In some embodiments, the oil is a medium chain triglyceride (MCT). In some embodiments, the oil is sunflower oil. In some embodiments the oil is tocopherol oil. The MCT may be from any suitable source. In some embodiments, the MCT is a triglyceride having aliphatic tails containing six, seven, eight, nine, ten, eleven, or twelve carbons. In some embodiments, the MCT is derived from a natural source, such as coconut oil. In some embodiments, the oil is a coconut MCT oil. In some embodiments, the multi-biotic supplement contains the oil in an amount of about 10 mg to about 1000 mg. In some embodiments, the multi-biotic supplement contains the oil in an amount of about 10 mg to about 500 mg. In some embodiments, the multi-biotic supplement contains the oil in an amount of about 20 mg to about 300 mg. In some embodiments, the multi-biotic supplement contains the oil an amount of about 20 mg to about 200 mg. In some embodiments, the multi-biotic supplement contains oil in an amount of about 30 mg to about 150 mg. In some embodiments, the multi-biotic supplement contains the oil in an amount of about 40 mg to about 120 mg. In some embodiments, the multi-biotic supplement contains the oil in an amount of about 50 mg to about 110 mg. In some embodiments, the multi-biotic supplement contains the oil in an amount of about 60 mg to about 100 mg. In some embodiments, the multi-biotic supplement contains the oil in an amount of about 70 mg to about 100 mg. In some embodiments, the multi-biotic supplement contains the oil in an amount of about 75 mg to about 100 mg. In some embodiments, the multi-biotic supplement contains the oil in an amount of about 80 mg to about 90 mg. In some embodiments, the multi-biotic supplement contains the oil in an amount selected from about 20 mg, about 25 mg, about 50 mg, about 55 mg, about 60 mg, about 65 mg, about 75 mg, about 80 mg, about 85 mg, about 90, and about 95 mg. In some embodiments, the multi-biotic supplement contains the oil in an amount of about 85 mg. In some embodiments, the multi-biotic supplement contains MCT oil in an amount of about 85 mg. In some embodiments, the multi-biotic supplement contains coconut MCT oil in an amount of about 85 mg.

### B. Probiotics

Generally, the multi-biotic supplements described herein contain one or more probiotics. In some embodiments, the probiotic is a probiotic blend of two or more probiotics.

The one or more probiotics of some embodiments may include one or more spore-forming species, one or more non-spore-forming species, or any combination thereof. In some embodiments, the one or more probiotics are spore-forming species. In some embodiments, the one or more probiotics are non-spore-forming species. In some embodiments, the one or more probiotics contain one or more Bacillus species, or one or more Bacillus strains, or a combination thereof. In some embodiments, the one or more probiotics contain one or more Bifidobacterium species, or one or more Bifidobacterium strains, or a combination thereof. In some embodiments, the one or more probiotics contain a Bifidobacterium animalis species. In some embodiments, the one or more probiotics contain one or more Saccharomyces species, or one or more Saccharomyces strains, or a combination thereof. In some embodiments, the one or more probiotics contain one or more Streptococcus species, or one or more Streptococcus strains, or a combination thereof. In some embodiments, the one or more probiotics contain one or more Pediococcus species, or one or more Pediococcus strains, or a combination thereof. In some embodiments, the one or more probiotics contain one or more Leuconostoc species, or one or more Leuconostocs strains, or a combination thereof. In some embodiments, the one or more probiotics contain one or more Escherichia species, or one or more Escherichia strains, or a combination thereof. In some embodiments, the one or more probiotics contain Bifidobacterium animalis ssp. lactis BB-12®. In some embodiments, the one or more probiotics contain one or more Lactobacillus species, one or more Lactobacillus strains, or a combination thereof. In some embodiments, the one or more probiotics contain a Lactobacillus rhamnosus species. In some embodiments, the one or more probiotics contain Lactobacillus rhamnosus GG. In some embodiments, the probiotic is a probiotic blend that contains one or more Bifidobacterium species or strains and one or more Lactobacillus species or strains. In some embodiments, the probiotic blend contains Bifidobacterium animalis ssp. lactis BB-12 and Lactobacillus rhamnosus GG.

In some embodiments, the multi-biotic supplement contains the one or more probiotics in an amount of about 1 mg to about 1000 mg. In some embodiments, the multi-biotic supplement contains the one or more probiotics in an amount of about 5 mg to about 800 mg. In some embodiments, the multi-biotic supplement contains the one or more probiotics in an amount of about 10 mg to about 600 mg. In some embodiments, the multi-biotic supplement contains the one or more probiotics in an amount of about 20 mg to about 500 mg. In some embodiments, the multi-biotic supplement contains the one or more probiotics in an amount of about 30 mg to about 400 mg. In some embodiments, the multi-biotic supplement contains the one or more probiotics in an amount of about 40 mg to about 300 mg. In some embodiments, the multi-biotic supplement contains the one or more probiotics in an amount of about 50 mg to about 200 mg. In some embodiments, the multi-biotic supplement contains the one or more probiotics in an amount of about 60 mg to about 180 mg. In some embodiments, the multi-biotic supplement contains the one or more probiotics in an amount of about 70 mg to about 160 mg. In some embodiments, the multi-biotic supplement contains the one or more probiotics in an amount of about 80 mg to about 150 mg. In some embodiments, the multi-biotic supplement contains the one or more probiotics in an amount of about 90 mg to about 150 mg. In some embodiments, the multi-biotic supplement contains the one or more probiotics in an amount of about 100 mg to about 150 mg. In some embodiments, the multi-biotic supplement contains the one or more probiotics in an amount of about 100 mg to about 140 mg. In some embodiments, the multi-biotic supplement contains the one or more probiotics in an amount of about 110 mg to about 130 mg. In some embodiments, the multi-biotic supplement contains the one or more probiotics in an amount selected from about 1 mg, about 5 mg, about 10 mg, about 15 mg, about 20 mg, about 25 mg, about 30 mg, about 35 mg, about 40 mg, about 50 mg, about 60 mg, about 70 mg, about 80 mg, about 90 mg, 100 mg, about 110 mg, about 120 mg, about 130 mg, and about 140 mg. In some embodiments, the multi-biotic supplement contains the one or more probiotics in an amount of about 120 mg.

The probiotic content of the multi-biotic supplement may be described in terms of bacterial counts in units of colony-forming units (CFUs). The bacterial counts of the multi-biotic supplements described herein may describe the count at the time of formulation and/or at the time of administration. It may be advantageous to formulate the supplements with a higher bacterial count with the understanding that the count per supplement may decrease over time prior to administration.

In some embodiments, the multi-biotic supplement has a total probiotic content of about 100 million CFUs to about 100 billion CFUs. In some embodiments, the multi-biotic supplement has a total probiotic content of about 500 million CFUs to about 80 billion CFUs. In some embodiments, the multi-biotic supplement has a total probiotic content of about 1 billion CFUs to about 70 billion CFUs. In some embodiments, the multi-biotic supplement has a total probiotic content of about 2 billion CFUs to about 60 billion CFUs. In some embodiments, the multi-biotic supplement has a total probiotic content of about 5 billion CFUs to about 55 billion CFUs. In some embodiments, the multi-biotic supplement has a total probiotic content of about 10 billion CFUs to about 50 billion CFUs. In some embodiments, the multi-biotic supplement has a total probiotic content of about 15 billion CFUs to about 45 billion CFUs. In some embodiments, the multi-biotic supplement has a total probiotic content of about 20 billion CFUs to about 40 billion CFUs. In some embodiments, the multi-biotic supplement has a total probiotic content of about 25 billion CFUs to about 40 billion CFUs. In some embodiments, the multi-biotic supplement has a total probiotic content of about 25 billion CFUs to about 35 billion CFUs. In some embodiments, the multi-biotic supplement has a total probiotic content of about 30 billion CFUs to about 35 billion CFUs. In some embodiments, the multi-biotic supplement has a total probiotic content of about 28 billion CFUs, about 30 billion CFUs, about 32 billion CFUs, or about 34 billion CFUs. In some embodiments, the multi-biotic supplement has a total probiotic content of about 32 billion CFUs.

In some embodiments, the multi-biotic supplement has a total probiotic content of about 1 billion CFUs to about 25 billion CFUs. In some embodiments, the multi-biotic supplement has a total probiotic content of about 2 billion CFUs to about 20 billion CFUs. In some embodiments, the multi-biotic supplement has a total probiotic content of about 5 billion CFUs to about 15 billion CFUs. In some embodiments, the multi-biotic supplement has a total probiotic content of about 8 billion CFUs to about 15 billion CFUs. In some embodiments, the multi-biotic supplement has a total probiotic content of about 9 billion CFUs to about 13 billion CFUs. In some embodiments, the multi-biotic supplement has a total probiotic content of about 10 billion CFUs to about 12 billion CFUs. In some embodiments, the multi-biotic supplement has a total probiotic content of about 9 billion CFUs, about 10 billion CFUs, about 11 billion CFUs, or about 12 billion CFUs. In some embodiments, the multi-biotic supplement has a total probiotic content of about 11 billion CFUs.

In some embodiments, the multi-biotic supplement contains Bifidobacterium animalis ssp. lactis BB-12 in an amount of about 100 million CFUs to about 5 billion CFUs. In some embodiments, the multi-biotic supplement contains Bifidobacterium animalis ssp. lactis BB-12 in an amount of about 200 million CFUs to about 2 billion CFUs. In some embodiments, the multi-biotic supplement contains Bifidobacterium animalis ssp. lactis BB-12 in an amount of about 500 million CFUs to about 1.5 billion CFUs. In some embodiments, the multi-biotic supplement contains Bifidobacterium animalis ssp. lactis BB-12 in an amount of about 600 million CFUs to about 1.4 billion CFUs. In some embodiments, the multi-biotic supplement contains Bifidobacterium animalis ssp. lactis BB-12 in an amount of about 800 million CFUs to about 1.2 billion CFUs. In some embodiments, the multi-biotic supplement contains Bifidobacterium animalis ssp. lactis BB-12 in an amount of about 900 million CFUs to about 1.1 billion CFUs. In some embodiments, the multi-biotic supplement contains Bifidobacterium animalis ssp. lactis BB-12 in an amount of about 900 million CFUs, about 1 billion CFUs, about 1.1 billion CFUs, or about 1.2 billion CFUs. In some embodiments, the multi-biotic supplement contains Bifidobacterium animalis ssp. lactis BB-12 in an amount of about 1 billion CFUs.

In some embodiments, the multi-biotic supplement contains Lactobacillus rhamnosus GG in an amount of about 500 million CFUs to about 20 billion CFUs. In some embodiments, the multi-biotic supplement contains Lactobacillus rhamnosus GG in an amount of about 1 billion CFUs to about 20 billion CFUs. In some embodiments, the multi-biotic supplement contains Lactobacillus rhamnosus GG in an amount of about 4 billion CFUs to about 16 billion CFUs. In some embodiments, the multi-biotic supplement contains Lactobacillus rhamnosus GG in an amount of about 6 billion CFUs to about 14 billion CFUs. In some embodiments, the multi-biotic supplement contains Lactobacillus rhamnosus GG in an amount of about 8 billion CFUs to about 12 billion CFUs. In some embodiments, the multi-biotic supplement contains Lactobacillus rhamnosus GG in an amount of about 9 million CFUs to about 11 billion CFUs. In some embodiments, the multi-biotic supplement contains Lactobacillus rhamnosus GG in an amount of about 9 billion CFUs, about 10 billion CFUs, or about 11 billion CFUs. In some embodiments, the multi-biotic supplement contains Lactobacillus rhamnosus GG in an amount of about 10 billion CFUs.

In some embodiments, the multi-biotic supplement contains colony forming units of Lactobacillus rhamnosus GG and Bifidobacterium animalis ssp. lactis BB-12 in a ratio ranging from about 1:1 to about 100:1. In some embodiments, the multi-biotic supplement contains colony forming units of Lactobacillus rhamnosus GG and Bifidobacterium animalis ssp. lactis BB-12 in a ratio ranging from about 2:1 to about 50:1. In some embodiments, the multi-biotic supplement contains colony forming units of Lactobacillus rhamnosus GG and Bifidobacterium animalis ssp. lactis BB-12 in a ratio ranging from about 3:1 to about 30:1. In some embodiments, the multi-biotic supplement contains colony forming units of Lactobacillus rhamnosus GG and Bifidobacterium animalis ssp. lactis BB-12 in a ratio ranging from about 5:1 to about 20:1. In some embodiments, the multi-biotic supplement contains colony forming units of Lactobacillus rhamnosus GG and Bifidobacterium animalis ssp. lactis BB-12 in a ratio ranging from about 8:1 to about 12:1. In some embodiments, the multi-biotic supplement contains colony forming units of Lactobacillus rhamnosus GG and Bifidobacterium animalis ssp. lactis BB-12 in a ratio of about 8:1, about 9:1, about 10:1, about 11:1, or about 12:1. In some embodiments, the multi-biotic supplement contains colony forming units of Lactobacillus rhamnosus GG and Bifidobacterium animalis ssp. lactis BB-12 in a ratio of about 10:1.

### C. Prebiotics

Generally, the multi-biotic supplements of this disclosure contain one or more prebiotics. In some embodiments, the prebiotic is a prebiotic blend of two or more prebiotics. In some embodiments, the prebiotic is a prebiotic blend of three or more prebiotics. In some embodiments, the prebiotic is a prebiotic blend of four or more prebiotics.

The one or more prebiotics of some embodiments may include one or more bacteriophage. The one or more prebiotics of some embodiments may include two or more bacteriophage. The one or more prebiotics of some embodiments may include three or more bacteriophage. The one or more prebiotics of some embodiments may include four or more bacteriophage. In some embodiments, the one or more bacteriophage include one or more Myoviridae species. In some embodiments, the one or more bacteriophage include one or more Myoviridae species selected from LHO1-Myoviridae, T4D-Myoviridae, and LL12-Myoviridae. In some embodiments, the one or more bacteriophage include LHO1-Myoviridae. In some embodiments, the one or more bacteriophage include T4D-Myoviridae. In some embodiments, the one or more bacteriophage include LL12-Myoviridae. In some embodiments, the one or more bacteriophage include one or more Siphoviridae species. In some embodiments, the one or more bacteriophage include LL5-Siphoviridae. In some embodiments, the prebiotic is a prebiotic blend that includes two or more Myoviridae species. In some embodiments, the prebiotic is a prebiotic blend that includes three or more Myoviridae species. In some embodiments, the prebiotic is a prebiotic blend that includes one or more Myoviridae species and one or more Siphoviridae species. In some embodiments, the prebiotic is a prebiotic blend that includes two or more Myoviridae species and one or more Siphoviridae species. In some embodiments, the prebiotic is a prebiotic blend that includes three or more Myoviridae species and one or more Siphoviridae species. In some embodiments, the prebiotic blend includes two or more selected from LHO1-Myoviridae, LL5-Siphoviridae, T4D-Myoviridae, and LL12-Myoviridae. In some embodiments, the prebiotic blend includes three or more selected from LH01-Myoviridae, LL5-Siphoviridae, T4D-Myoviridae, and LL12-Myoviridae. In some embodiments, the prebiotic blend includes LHO1-Myoviridae, LL5-Siphoviridae, T4D-Myoviridae, and LL12-Myoviridae. It should be understood that a prebiotic is any non-digestible food ingredient that beneficially affects the host by selectively stimulating the growth and/or activity of one or a limited number of bacteria in the colon.

In some embodiments, the multi-biotic supplement contains the prebiotic in an amount of about 0.5 mg to about 500 mg. In some embodiments, the multi-biotic supplement contains the prebiotic in an amount of about 1 mg to about 100 mg. In some embodiments, the multi-biotic supplement contains the prebiotic in an amount of about 1 mg to about 75 mg. In some embodiments, the multi-biotic supplement contains the prebiotic in an amount of about 1 mg to about 50 mg. In some embodiments, the multi-biotic supplement contains the prebiotic in an amount of about 2 mg to about 50 mg. In some embodiments, the multi-biotic supplement contains the prebiotic in an amount of about 2 mg to about 40 mg. In some embodiments, the multi-biotic supplement contains the prebiotic in an amount of about 2 mg to about 30 mg. In some embodiments, the multi-biotic supplement contains the prebiotic in an amount of about 3 mg to about 25 mg. In some embodiments, the multi-biotic supplement contains the prebiotic in an amount of about 5 mg to about 20 mg. In some embodiments, the multi-biotic supplement contains the prebiotic in an amount of about 10 mg to about 20 mg. In some embodiments, the multi-biotic supplement contains the prebiotic in an amount selected from about 10 mg, about 12 mg, about 15 mg, about 17 mg, and about 20 mg. In some embodiments, the multi-biotic supplement contains the prebiotic in an amount of about 15 mg.

In some embodiments, the multi-biotic supplement contains one or more bacteriophages in an amount of about 0.5 mg to about 500 mg. In some embodiments, the multi-biotic supplement contains one or more bacteriophages in an amount of about 1 mg to about 100 mg. In some embodiments, the multi-biotic supplement contains one or more bacteriophages in an amount of about 1 mg to about 75 mg. In some embodiments, the multi-biotic supplement contains one or more bacteriophages in an amount of about 1 mg to about 50 mg. In some embodiments, the multi-biotic supplement contains one or more bacteriophages in an amount of about 2 mg to about 50 mg. In some embodiments, the multi-biotic supplement contains one or more bacteriophages in an amount of about 2 mg to about 40 mg. In some embodiments, the multi-biotic supplement contains one or more bacteriophages in an amount of about 2 mg to about 30 mg. In some embodiments, the multi-biotic supplement contains one or more bacteriophages in an amount of about 3 mg to about 25 mg. In some embodiments, the multi-biotic supplement contains one or more bacteriophages in an amount of about 5 mg to about 20 mg. In some embodiments, the multi-biotic supplement contains one or more bacteriophages in an amount of about 10 mg to about 20 mg. In some embodiments, the multi-biotic supplement contains one or more bacteriophages in an amount selected from about 10 mg, about 12 mg, about 15 mg, about 17 mg, and about 20 mg. In some embodiments, the multi-biotic supplement contains one or more bacteriophages in an amount of about 15 mg. In some embodiments, the multi-biotic supplement contains one or more bacteriophages in an amount of about 15 mg, wherein the one or more bacteriophages include Myoviridae species selected from LH01-Myoviridae, T4D-Myoviridae, and/or LL12-Myoviridae.

### D. Relative Amounts of Postbiotics or Postbiotic Precursors, Probiotics, and Prebiotics

In some embodiments, the multi-biotic supplement contains a postbiotic and a prebiotic in amounts such that a weight ratio of the postbiotic to the prebiotic ranges from about 1:2 to about 100:1. In some embodiments, the multi-biotic supplement contains a postbiotic and a prebiotic in amounts such that a weight ratio of the postbiotic to the prebiotic ranges from about 1:1 to about 100:1. In some embodiments, the multi-biotic supplement contains a postbiotic and a prebiotic in amounts such that a weight ratio of the postbiotic to the prebiotic ranges from about 1:1 to about 50:1. In some embodiments, the multi-biotic supplement contains a postbiotic and a prebiotic in amounts such that a weight ratio of the postbiotic to the prebiotic ranges from about 1:1 to about 40:1. In some embodiments, the multi-biotic supplement contains a postbiotic and a prebiotic in amounts such that a weight ratio of the postbiotic to the prebiotic ranges from about 5:1 to about 50:1. In some embodiments, the multi-biotic supplement contains a postbiotic and a prebiotic in amounts such that a weight ratio of the postbiotic to the prebiotic ranges from about 5:1 to about 40:1. In some embodiments, the multi-biotic supplement contains a postbiotic and a prebiotic in amounts such that a weight ratio of the postbiotic to the prebiotic ranges from about 10:1 to about 30:1. In some embodiments, the multi-biotic supplement contains a postbiotic and a prebiotic in amounts such that a weight ratio of the postbiotic to the prebiotic ranges from about 15:1 to about 25:1. In some embodiments, the multi-biotic supplement contains a postbiotic and a prebiotic in amounts such that a weight ratio of the postbiotic to the prebiotic ranges from about 20:1 to about 30:1. In some embodiments, the multi-biotic supplement contains a postbiotic and a prebiotic in amounts such that a weight ratio of the postbiotic to the prebiotic is about 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, or 100:1. In some embodiments, the multi-biotic supplement contains a postbiotic and a prebiotic in amounts such that a weight ratio of the postbiotic to the prebiotic is about 18:1, about 20:1, about 22:1, about 23:1, about 24:1, or about 25:1. In some embodiments, the multi-biotic supplement contains a postbiotic and a prebiotic in amounts such that a weight ratio of the postbiotic to the prebiotic is about 20:1. In some embodiments, the multi-biotic supplement contains a postbiotic and a prebiotic in amounts such that a weight ratio of the postbiotic to the prebiotic is about 23:1.

In some embodiments, the multi-biotic supplement contains a postbiotic precursor and a prebiotic in amounts such that a weight ratio of the postbiotic precursor to the prebiotic ranges from about 1:2 to about 100:1. In some embodiments, the multi-biotic supplement contains a postbiotic and a prebiotic in amounts such that a weight ratio of the postbiotic precursor to the prebiotic precursor ranges from about 1:1 to about 100:1. In some embodiments, the multi-biotic supplement contains a postbiotic precursor and a prebiotic in amounts such that a weight ratio of the postbiotic precursor to the prebiotic ranges from about 1:1 to about 50:1. In some embodiments, the multi-biotic supplement contains a postbiotic and a prebiotic in amounts such that a weight ratio of the postbiotic precursor to the prebiotic precursor ranges from about 1:1 to about 40:1. In some embodiments, the multi-biotic supplement contains a postbiotic precursor and a prebiotic in amounts such that a weight ratio of the postbiotic precursor to the prebiotic ranges from about 5:1 to about 50:1. In some embodiments, the multi-biotic supplement contains a postbiotic precursor and a prebiotic in amounts such that a weight ratio of the postbiotic precursor to the prebiotic ranges from about 5:1 to about 40:1. In some embodiments, the multi-biotic supplement contains a postbiotic precursor and a prebiotic in amounts such that a weight ratio of the postbiotic precursor to the prebiotic ranges from about 10:1 to about 30:1. In some embodiments, the multi-biotic supplement contains a postbiotic precursor and a prebiotic in amounts such that a weight ratio of the postbiotic precursor to the prebiotic ranges from about 15:1 to about 25:1. In some embodiments, the multi-biotic supplement contains a postbiotic precursor and a prebiotic in amounts such that a weight ratio of the postbiotic precursor to the prebiotic ranges from about 20:1 to about 30:1. In some embodiments, the multi-biotic supplement contains a postbiotic precursor and a prebiotic precursor in amounts such that a weight ratio of the postbiotic to the prebiotic is about 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, or 100:1. In some embodiments, the multi-biotic supplement contains a postbiotic precursor and a prebiotic in amounts such that a weight ratio of the postbiotic precursor to the prebiotic is about 18:1, about 20:1, about 22:1, about 23:1, about 24:1, or about 25:1. In some embodiments, the multi-biotic supplement contains a postbiotic precursor and a prebiotic in amounts such that a weight ratio of the postbiotic precursor to the prebiotic is about 20:1. In some embodiments, the multi-biotic supplement contains a postbiotic precursor and a prebiotic in amounts such that a weight ratio of the postbiotic precursor to the prebiotic is about 23:1.

In some embodiments, the multi-biotic supplement contains a probiotic and a prebiotic in amounts such that a weight ratio of the probiotic to the prebiotic ranges from about 1:5 to about 50:1, about 1:4 to about 50:1, about 1:3 to about 50:1, about 1:2 to about 50:1, or about 1:1 to about 50:1. In some embodiments, the multi-biotic supplement contains a probiotic and a prebiotic in amounts such that a weight ratio of the probiotic to the prebiotic ranges from about 1:5 to about 30:1, about 1:4 to about 30:1, about 1:3 to about 30:1, about 1:2 to about 30:1, or about 1:1 to about 30:1. In some embodiments, the multi-biotic supplement contains a probiotic and a prebiotic in amounts such that a weight ratio of the probiotic to the prebiotic ranges from about 1:5 to about 20:1, about 1:4 to about 20:1, about 1:3 to about 20:1, about 1:2 to about 20:1, or about 1:1 to about 20:1. In some embodiments, the multi-biotic supplement contains a probiotic and a prebiotic in amounts such that a weight ratio of the probiotic to the prebiotic ranges from about 2:1 to about 20:1. In some embodiments, the multi-biotic supplement contains a probiotic and a prebiotic in amounts such that a weight ratio of the probiotic to the prebiotic ranges from about 2:1 to about 15:1. In some embodiments, the multi-biotic supplement contains a probiotic and a prebiotic in amounts such that a weight ratio of the probiotic to the prebiotic ranges from about 4:1 to about 12:1. In some embodiments, the multi-biotic supplement contains a probiotic and a prebiotic in amounts such that a weight ratio of the probiotic to the prebiotic ranges from about 6:1 to about 10:1. In some embodiments, the multi-biotic supplement contains a probiotic and a prebiotic in amounts such that a weight ratio of the probiotic to the prebiotic is about 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, or 100:1. In some embodiments, the multi-biotic supplement contains a probiotic and a prebiotic in amounts such that a weight ratio of the probiotic to the prebiotic is about 8:1.

In some embodiments, the multi-biotic supplement contains a postbiotic and a probiotic in amounts such that a weight ratio of the postbiotic to the probiotic ranges from about 1:2 to about 10:1. In some embodiments, the multi-biotic supplement contains a postbiotic and a probiotic in amounts such that a weight ratio of the postbiotic to the probiotic ranges from about 1:1 to about 10:1. In some embodiments, the multi-biotic supplement contains a postbiotic and a probiotic in amounts such that a weight ratio of the postbiotic to the probiotic ranges from about 1:1 to about 8:1. In some embodiments, the multi-biotic supplement contains a postbiotic and a probiotic in amounts such that a weight ratio of the postbiotic to the probiotic ranges from about 1:1 to about 5:1. In some embodiments, the multi-biotic supplement contains a postbiotic and a probiotic in amounts such that a weight ratio of the postbiotic to the probiotic ranges from about 2:1 to about 5:1. In some embodiments, the multi-biotic supplement contains a postbiotic and a probiotic in amounts such that a weight ratio of the postbiotic to the probiotic ranges from about 2:1 to about 4:1. In some embodiments, the multi-biotic supplement contains a postbiotic and a probiotic in amounts such that a weight ratio of the postbiotic to the probiotic is about 1:2, 1:1, 2:1, 3:1, 4:1, 5:1, 6:1, 7:1, 8:1, 9:1, or 10:1. In some embodiments, the multi-biotic supplement contains a postbiotic and a probiotic in amounts such that a weight ratio of the postbiotic to the probiotic is about 3:1.

In some embodiments, the multi-biotic supplement contains a postbiotic precursor and a probiotic in amounts such that a weight ratio of the postbiotic precursor to the probiotic ranges from about 1:2 to about 10:1. In some embodiments, the multi-biotic supplement contains a postbiotic precursor and a probiotic in amounts such that a weight ratio of the postbiotic precursor to the probiotic ranges from about 1:1 to about 10:1. In some embodiments, the multi-biotic supplement contains a postbiotic precursor and a probiotic in amounts such that a weight ratio of the postbiotic precursor to the probiotic ranges from about 1:1 to about 8:1. In some embodiments, the multi-biotic supplement contains a postbiotic precursor and a probiotic in amounts such that a weight ratio of the postbiotic precursor to the probiotic ranges from about 1:1 to about 5:1. In some embodiments, the multi-biotic supplement contains a postbiotic precursor and a probiotic in amounts such that a weight ratio of the postbiotic precursor to the probiotic ranges from about 2:1 to about 5:1. In some embodiments, the multi-biotic supplement contains a postbiotic precursor and a probiotic in amounts such that a weight ratio of the postbiotic precursor to the probiotic ranges from about 2:1 to about 4:1. In some embodiments, the multi-biotic supplement contains a postbiotic precursor and a probiotic in amounts such that a weight ratio of the postbiotic precursor to the probiotic is about 1:2, 1:1, 2:1, 3:1, 4:1, 5:1, 6:1, 7:1, 8:1, 9:1, or 10:1. In some embodiments, the multi-biotic supplement contains a postbiotic precursor and a probiotic in amounts such that a weight ratio of the postbiotic precursor to the probiotic is about 3:1.

### E. Additional Components

In some embodiments, the multi-biotic supplement may further include one or more additional ingredients. In some embodiments, the additional ingredients may be selected from cellulosic materials, citric acid, starches, proteins, alginates, calcium phosphate salts (such as tricalcium phosphate and dicalcium phosphate), lubricants, lecithins, and silicon dioxide. In some embodiments, the cellulosic material may be selected from microcrystalline cellulose and hydroxypropyl methylcellulose. In some embodiments, the lubricant may be a stearate. In some embodiments, the lubricant may be magnesium stearate. In some embodiments, the multi-biotic supplement may include one or more selected from a cellulosic material, a lubricant, and silicon dioxide. In some embodiments, the multi-biotic supplement may include two or more selected from a cellulosic material, a lubricant, and silicon dioxide. In some embodiments, the multi-biotic supplement may contain a cellulosic material, a lubricant, and silicon dioxide. In some embodiments, the multi-biotic supplement may include microcrystalline cellulose, hydroxypropyl methylcellulose, magnesium stearate, and silicon dioxide.

In some embodiments, the multi-biotic supplement may contain one or more excipients. In some embodiments, the one or more excipients may, for example, be one or more binders, fillers, diluents, lubricants, or viscosity adjusters. In some embodiments, the excipient may be one or more selected from ascorbyl palmitate, silicon dioxide, rice dextrin, leucine, cellulose, oils (e.g., plant oils, coconut oil, sunflower oil, vitamin E oil), silicon dioxide, rice leucine, hypromellose (HPMC), and MCTs. In some embodiments, the plant oils may include coconut oil or sunflower oil. In some embodiments, the excipient may be silicon dioxide.

In some embodiments, the multi-biotic supplement contains one or more excipients (e.g., together, or independently) in an amount of about 1 mg to about 500 mg. In some embodiments, the multi-biotic supplement contains the excipient in an amount of about 1 mg to about 100 mg. In some embodiments, the multi-biotic supplement contains the excipient in an amount of about 1 mg to about 75 mg. In some embodiments, the multi-biotic supplement contains the excipient in an amount of about 1 mg to about 50 mg. In some embodiments, the multi-biotic supplement contains the excipient in an amount of about 2 mg to about 50 mg. In some embodiments, the multi-biotic supplement contains the excipient in an amount of about 5 mg to about 40 mg. In some embodiments, the multi-biotic supplement contains the excipient in an amount of about 10 mg to about 30 mg. In some embodiments, the multi-biotic supplement contains the excipient in an amount of about 15 mg to about 25 mg. In some embodiments, the multi-biotic supplement contains the excipient in an amount of about 50 mg to about 500 mg, about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, about 450 mg to about 500 mg, about 50 mg to about 450 mg, about 100 mg to about 450 mg, about 150 mg to about 450 mg, about 200 mg to about 450 mg, about 250 mg to about 450 mg, about 300 mg to about 450 mg, about 350 mg to about 450 mg, about 400 mg to about 450 mg, about 50 mg to about 400 mg, about 100 mg to about 400 mg, about 150 mg to about 400 mg, about 200 mg to about 400 mg, about 250 mg to about 400 mg, about 300 mg to about 400 mg, about 350 mg to about 400 mg, about 50 mg to about 400 mg, about 100 mg to about 400 mg, about 150 mg to about 400 mg, about 200 mg to about 400 mg, about 250 mg to about 400 mg, about 300 mg to about 400 mg, about 50 mg to about 350 mg, about 100 mg to about 350 mg, about 150 mg to about 350 mg, about 200 mg to about 350 mg, about 250 mg to about 350 mg, about 300 mg to about 350 mg, about 50 mg to about 300 mg, about 100 mg to about 300 mg, about 150 mg to about 300 mg, about 200 mg to about 300 mg, about 250 mg to about 300 mg, about 50 mg to about 250 mg, about 100 mg to about 250 mg, about 150 mg to about 250 mg, about 200 mg to about 250 mg, about 50 mg to about 200 mg, about 100 mg to about 200 mg, about 150 mg to about 200 mg, about 50 mg to about 150 mg, about 100 mg to about 150 mg, or about 50 mg to about 100 mg. In some embodiments, the multi-biotic supplement contains the excipient in an amount selected from about 15 mg, about 18 mg, about 20 mg, about 22 mg, and about 25 mg. In some embodiments, the multi-biotic supplement contains the excipient in an amount of about 10, 20, 30, 40, 50, 60, 70, 80, 90, 100, 150, 200, 250, 300, 250, 400, 450, or 500. In some embodiments, the multi-biotic supplement contains the excipient in an amount of about 20 mg.

### Multi-Biotic Supplement Capsules

The multi-biotic supplements provided herein may be produced by any suitable methods known in the art. They may be produced from any suitable materials known in the art. In some embodiments, the multi-biotic supplement may be administered as a single dose or multiple doses. In some embodiments, the multi-biotic supplement may be administered as multiple doses, where each dose includes different components of the supplement or the same components of the supplement. The multi-biotic supplements provided herein may be produced as one or more gummies, tablets, soft-gels, or powders. In some embodiments, the multi-biotic supplement may be contained in one or more capsules. In some embodiments, the multi-biotic supplement is contained in two capsules. In some embodiments, the multi-biotic supplement is contained in one capsule. In some embodiments, the multi-biotic supplement is a single dose that contains more than one capsules. In some embodiments, the multi-biotic supplement is contained in one capsule. In some embodiments, the multi-biotic supplement is a single dose that contains two capsules. In some embodiments, the multi-biotic supplement is a single dose that contains both an outer capsule and an inner capsule.

In some embodiments, a single dose contains both an outer capsule and an inner capsule.

### A. Outer Capsule

In some embodiments, the outer capsule may be an extended-release capsule that delays or extends the release of the capsule contents. For example, extended-release capsules may reduce the amount of capsule contents released in the stomach of a subject, such that the majority or all of the contents are released after the capsule passes through the stomach. In some embodiments, the outer capsule of some embodiments may be configured to bypass the stomach of the subject. In some embodiments, the outer capsule is time dependent, releasing the ingredients after at least 10, 20, 30, 40, 50, 60, 70, 80, 90, or 100 or more minutes after ingestion. In some embodiments, the outer capsule of some embodiments may be configured to release its contents after entering the intestine of the subject. In some embodiments, the outer capsule of some embodiments may be configured to release its contents after entering the small intestine of the subject. In some embodiments, the outer capsule of some embodiments may be configured to bypass the stomach of the subject and release its contents after entering the intestine of the subject. In some embodiments, the outer capsule of some embodiments may be configured to bypass the stomach of the subject and release its contents after entering the small intestine of the subject.

In some embodiments, the use of a delayed release capsule may increase absorption of the supplement's components contained therein, decrease unwanted interactions of the supplement's components contained therein, or both, by releasing the supplement's components where they may be absorbed in the gastrointestinal tract. Bypassing the stomach may prevent exposure of the supplement components to the low pH of the stomach, which may cause them to undergo one or more chemical reactions. In addition, stomach acid can cause unwanted interactions between one or more components that do not occur in the small intestine. In some embodiments, the use of delayed release design for the outer capsule helps protect the inner capsule to remain intact toward the end of the upper gastrointestinal tract. In some embodiments the use of a delayed release capsule helps components of inner capsules such as probiotics survive the harsh conditions such as stomach acid and bile of the upper gastrointestinal tract for delivery to the colon, an ideal place for probiotics to grow and thrive. In some embodiments the delayed release capsule is part of a colon-targeted probiotic delivery system.

The outer capsule may be any size suitable for containing the components of the multi-biotic supplement. In some embodiments, the multi-biotic supplement may have an outer capsule that is size 00. In some embodiments, the outer capsule may comprise one or more polymers. In some embodiments, the one or more polymers may be selected from hydroxypropyl methylcellulose (hypromellose), gellan gum, and pullalan. In some embodiments, the outer capsule comprises hypromellose and gellan gum. In some embodiments, the outer capsule comprises hypromellose, and gellan gum. In some embodiments, the outer capsule consists essentially of hypromellose, and gellan gum.

In some embodiments, the outer capsule is essentially free of ingredients derived from animals. In some embodiments, the outer capsule may contain less than 1 wt %, less than 0.1 wt %, or less than 0.01 wt % gelatin. In some embodiments, the outer capsule may be essentially free of gelatin.

In some embodiments, the outer capsule contains the postbiotic or postbiotic precursor described above. In some embodiments, the outer capsule contains one or more of the excipients described above. In some embodiments, the outer capsule contains one or more of the additional ingredients described above. In some embodiments, the outer capsule contains an inner capsule. In some embodiments, the outer capsule contains the postbiotic or postbiotic precursor described above, one or more of the excipients described above, and an inner capsule. In some embodiments, the outer capsule contains tributyrin, coconut MCT oil, and silicon dioxide. In some embodiments, the outer capsule contains tributyrin, coconut MCT oil, silicon dioxide, and an inner capsule.

### B. Inner Capsule

In some embodiments, the inner capsule is an immediate-release capsule that quickly releases the capsule contents. For instance, an immediate-release capsule may dissolve/disintegrate in less than about 30 minutes, 25 minutes, 20 minutes, 15 minutes, 10 minutes, or 5 minutes or less. In some embodiments, the multi-biotic supplement may be configured so as to reduce the amount of inner capsule contents released in the stomach and intestine of a subject, such that the majority or all of the contents are released after the capsule passes through the intestine. An immediate-release capsule may, for instance, be paired with a delayed-release capsule (e.g., delayed-release capsule residing within an extended release capsule, an extended-release capsule residing within a delayed-release capsule). In some embodiments, the multi-biotic supplement may be configured such that the inner capsule bypasses the stomach and the small intestine of the subject. In some embodiments, the inner capsule of some embodiments may be configured to release its contents after entering the colon of the subject.

The inner capsule may be any size suitable for containing the remaining components of the multi-biotic supplement and for encapsulation by the outer capsule. In some embodiments, the multi-biotic supplement may have an inner capsule that is size 3. In some embodiments, the inner capsule may comprise one or more polymers. In some embodiments, the one or more polymers may be selected from hydroxypropyl methylcellulose (hypromellose), gellan gum, and pullalan. In some embodiments, the inner capsule may comprise hypromellose. In some embodiments, the inner capsule may comprise hypromellose and silicon dioxide. In some embodiments, the inner capsule may consist essentially of hypromellose and silicon dioxide.

In some embodiments, the inner capsule is essentially free of ingredients derived from animals. In some embodiments, the inner capsule may contain less than 1 wt %, less than 0.1 wt %, or less than 0.01 wt % gelatin. In some embodiments, the inner capsule may be essentially free of gelatin.

In some embodiments, the inner capsule contains a probiotic described above. In some embodiments, the inner capsule contains a prebiotic described above. In some embodiments, the inner capsule contains one or more of the excipients described above. In some embodiments, the inner capsule contains one or more of an additional ingredient described above. In some embodiments, the inner capsule contains a probiotic described above and a prebiotic described above. In some embodiments, the inner capsule contains a probiotic described above, the prebiotic described above, and one or more of an excipient described above. In some embodiments, the inner capsule contains a probiotic described above, a prebiotic described above, and one or more of an additional ingredient described above. In some embodiments, the inner capsule contains a probiotic described above, a prebiotic described above, one or more of an excipient described above, and one or more of an additional ingredient described above. In some embodiments, the inner capsule comprises one or more of Bifidobacterium animalis ssp. lactis BB-12, Lactobacillus rhamnosus GG, LH01 Myoviridae, LL5-Siphoviridae, T4D-Myoviridae, and LL12-Myoviridae. In some embodiments, the inner capsule comprises one or more of Bifidobacterium animalis ssp. lactis BB-12, Lactobacillus rhamnosus GG, LH01-Myoviridae, LL5-Siphoviridae, T4D Myoviridae, LL12-Myoviridae, and one or more excipients selected from hypromellose, microcrystalline cellulose, magnesium stearate, and silicon dioxide.

In some embodiments, the outer capsule contains liquid components and the inner capsule contains dry ingredients. In some embodiments, the outer capsule comprises a postbiotic or postbiotic precursor, and an inner capsule, and the inner capsule comprises a prebiotic and a probiotic. In some embodiments, the outer capsule comprises tributyrin, an oil, silicon dioxide, and an inner capsule, and the inner capsule comprises Bifidobacterium animalis ssp. lactis BB-12, Lactobacillus rhamnosus GG, LH01 Myoviridae, LL5-Siphoviridae, T4D Myoviridae, and LL12-Myoviridae.

### Exemplary Multi-Biotic Supplements

In some embodiments, the present disclosure provides a multi-biotic supplement comprising (a)(i) a postbiotic precursor; (ii) a probiotic blend comprising one or more probiotics; and/or (iii) a prebiotic blend comprising one or more prebiotics; and (b)(ii) at least one capsule.

In some embodiments, the present disclosure provides a multi-biotic supplement comprising (a)(i) a postbiotic precursor comprising tributyrin; (ii) a probiotic blend comprising one or more probiotics, wherein the one or more probiotics comprise one or more Bifidobacterium species or strains and one or more Lactobacillus species or strains; and (iii) a prebiotic blend comprising one or more prebiotics, wherein the one or more prebiotics are of the Myoviridae species, and (b) at least one capsule.

In some embodiments, the present disclosure provides a multi-biotic supplement comprising (a)(i) a postbiotic precursor comprising tributyrin; (ii) a probiotic blend comprising one or more probiotics, wherein the one or more probiotics comprise Bifidobacterium animalis ssp. lactis BB-12 and Lactobacillus rhamnosus GG; and (iii) a prebiotic blend comprising one or more prebiotics, wherein the one or more prebiotics are LH01 Myoviridae, LL5-Siphoviridae, T4D Myoviridae, and LL12 Myoviridae, and (b) at least one capsule.

In some embodiments, the multi-biotic supplement contains the postbiotic precursor in an amount of about 500 mg to about 1000 mg. In some embodiments, the multi-biotic supplement contains the postbiotic precursor in an amount of about 250 mg to about 350 mg. In some embodiments, the multi-biotic supplement contains the postbiotic precursor in an amount of about 300 mg. In some embodiments, the multi-biotic supplement contains the one or more probiotics in an amount of about 50 mg to about 200 mg. In some embodiments, the multi-biotic supplement contains the one or more probiotics in an amount of about 110 mg to about 130 mg. In some embodiments, the multi-biotic supplement contains the one or more probiotics in an amount of about 120 mg. In some embodiments, the multi-biotic supplement contains the one or more prebiotics in an amount of about 2 mg to about 50 mg. In some embodiments, the multi-biotic supplement contains the one or more prebiotics in an amount of about 10 mg to about 20 mg. In some embodiments, the multi-biotic supplement contains the one or more prebiotics in an amount of about 15 mg.

In some embodiments, the present disclosure provides a multi-biotic supplement comprising (a)(i) a postbiotic precursor in an amount of about 250 mg to about 350 mg; (ii) a probiotic blend comprising one or more probiotics in an amount of about 110 mg to about 130 mg; and/or (iii) a prebiotic blend comprising one or more prebiotics in an amount of about 10 mg to about 20 mg; and (b) at least one capsule.

In some embodiments, the present disclosure provides a multi-biotic supplement comprising (a)(i) a postbiotic precursor in an amount of about 300 mg; (ii) a probiotic blend comprising one or more probiotics in an amount of about 120 mg; and/or a (iii) prebiotic blend comprising one or more prebiotics in an amount of about 15 mg; and (b) at least one capsule.

In some embodiments, the present disclosure provides a multi-biotic supplement comprising (a)(i) a postbiotic precursor in an amount of about 250 mg to about 350 mg, wherein the postbiotic precursor comprises tributyrin; (ii) a probiotic blend comprising one or more probiotics in an amount of about 110 mg to about 130 mg, wherein the one or more probiotics comprise one or more Bifidobacterium species or strains and one or more Lactobacillus species or strains; and (iii) a prebiotic blend comprising one or more prebiotics in an amount of about 10 mg to about 20 mg, wherein the one or more prebiotics are of the Myoviridae species, and (b) at least one capsule.

In some embodiments, the present disclosure provides a multi-biotic supplement comprising (a)(i) a postbiotic precursor in an amount of about 300 mg, wherein the postbiotic precursor comprises tributyrin; (ii) a probiotic blend comprising one or more probiotics in an amount of about 120 mg, wherein the one or more probiotics comprise one or more Bifidobacterium species or strains and one or more Lactobacillus species or strains; and (iii) a prebiotic blend comprising one or more prebiotics in an amount of about 15 mg, wherein the one or more prebiotics are of the Myoviridae species, and (b) at least one capsule.

In some embodiments, the present disclosure provides a multi-biotic supplement comprising (a)(i) a postbiotic precursor in an amount of about 250 mg to about 350 mg, wherein the postbiotic precursor comprises tributyrin; (ii) a probiotic blend comprising one or more probiotics in an amount of about 110 mg to about 130 mg, wherein the one or more probiotics comprise Bifidobacterium animalis ssp. lactis BB-12 and Lactobacillus rhamnosus GG; and (iii) a prebiotic blend comprising one or more prebiotics in an amount of about 10 mg to about 20 mg, wherein the one or more prebiotics are LH01 Myoviridae, LL5-Siphoviridae, T4D-Myoviridae, and LL12-Myoviridae, and (b) at least one capsule.

In some embodiments, the present disclosure provides a multi-biotic supplement comprising (a)(i) a postbiotic precursor in an amount of about 300 mg, wherein the postbiotic precursor comprises tributyrin; (ii) a probiotic blend comprising one or more probiotics in an amount of about 120 mg, wherein the one or more probiotics comprise Bifidobacterium animalis ssp. lactis BB-12 and Lactobacillus rhamnosus GG; and (iii) a prebiotic blend comprising one or more prebiotics in an amount of about 15 mg, wherein the one or more prebiotics are LHO1-Myoviridae, LL5-Siphoviridae, T4D Myoviridae, and LL12-Myoviridae, and (b) at least one capsule.

In some embodiments, the multi-biotic supplement comprises at least two capsules. In some embodiments, the multi-biotic supplement comprises an inner capsule disposed within an outer capsule. In some embodiments, the outer capsule comprises the postbiotic precursor. In some embodiments, the outer capsule comprises the postbiotic precursor. In some embodiments, the inner capsule comprises the probiotic blend. In some embodiments, the inner capsule comprises the prebiotic blend. In some embodiments, the inner capsule comprises the probiotic blend and the prebiotic blend. In some embodiments, the outer capsule comprises the postbiotic precursor and the inner capsule comprises the probiotic blend. In some embodiments, the outer capsule comprises the postbiotic precursor and the inner capsule comprises the prebiotic blend. In some embodiments, the outer capsule comprises the postbiotic precursor and the inner capsule comprises the probiotic blend and the prebiotic blend.

In some embodiments, the present disclosure provides a multi-biotic supplement comprising an outer capsule comprising a postbiotic precursor and an inner capsule comprising a probiotic blend and a prebiotic blend; and wherein the inner capsule is disposed within the outer capsule.

In some embodiments, the present disclosure provides a multi-biotic supplement comprising an outer capsule comprising a postbiotic precursor in an amount of about 250 mg to about 350 mg; and an inner capsule comprising a probiotic blend comprising one or more probiotics in an amount of about 110 mg to about 130 mg, and a prebiotic blend comprising one or more prebiotics in an amount of about 10 mg to about 20 mg; and wherein the inner capsule is disposed within the outer capsule.

In some embodiments, the present disclosure provides a multi-biotic supplement comprising an outer capsule comprising a postbiotic precursor in an amount of about 300 mg; and an inner capsule comprising a probiotic blend comprising one or more probiotics in an amount of about 120 mg, and a prebiotic blend comprising one or more prebiotics in an amount of about 15 mg; and wherein the inner capsule is disposed within the outer capsule.

In some embodiments, the present disclosure provides a multi-biotic supplement comprising an outer capsule comprising a postbiotic precursor, wherein the postbiotic precursor comprises tributyrin; and an inner capsule comprising (i) a probiotic blend comprising one or more Bifidobacterium species or strains and one or more Lactobacillus species or strains; and (ii) a prebiotic blend comprising one or more prebiotics, wherein the one or more prebiotics are of the Myoviridae species, wherein the inner capsule is disposed within the outer capsule.

In some embodiments, the present disclosure provides a multi-biotic supplement comprising an outer capsule comprising a postbiotic precursor in an amount of about 250 mg to about 350 mg, wherein the postbiotic precursor comprises tributyrin; and an inner capsule comprising (i) a probiotic blend comprising one or more probiotics in an amount of about 110 mg to about 130 mg, wherein the one or more probiotics comprise one or more Bifidobacterium species or strains and one or more Lactobacillus species or strains; and (ii) a prebiotic blend comprising one or more prebiotics in an amount of about 10 mg to about 20 mg, wherein the one or more prebiotics are of the Myoviridae species, wherein the inner capsule is disposed within the outer capsule.

In some embodiments, the present disclosure provides a multi-biotic supplement comprising an outer capsule comprising a postbiotic precursor, wherein the postbiotic precursor comprises tributyrin; and an inner capsule comprising (i) a probiotic blend comprising Bifidobacterium animalis ssp. lactis BB-12 and Lactobacillus rhamnosus GG; and (ii) a prebiotic blend comprising LH01 Myoviridae, LL5-Siphoviridae, T4D Myoviridae, and LL12-Myoviridae, wherein the inner capsule is disposed within the outer capsule.

In some embodiments, the present disclosure provides a multi-biotic supplement comprising an outer capsule comprising a postbiotic precursor in an amount of about 250 mg to about 350 mg, wherein the postbiotic precursor comprises tributyrin; and an inner capsule comprising (i) a probiotic blend comprising one or more probiotics in an amount of about 110 mg to about 130 mg, wherein the one or more probiotics comprise Bifidobacterium animalis ssp. lactis BB-12 and Lactobacillus rhamnosus GG; and (ii) a prebiotic blend comprising one or more prebiotics in an amount of about 10 mg to about 20 mg, wherein the one or more prebiotics comprise LHO1 Myoviridae, LL5-Siphoviridae, T4D Myoviridae, and LL12-Myoviridae, wherein the inner capsule is disposed within the outer capsule.

In some embodiments, the present disclosure provides a multi-biotic supplement comprising an outer capsule comprising a postbiotic precursor in an amount of about 300 mg, wherein the postbiotic precursor comprises tributyrin; and an inner capsule comprising (i) a probiotic blend comprising one or more probiotics in an amount of about 120 mg, wherein the one or more probiotics comprise Bifidobacterium animalis ssp. lactis BB-12 and Lactobacillus rhamnosus GG; and (ii) a prebiotic blend comprising one or more prebiotics in an amount of about 15 mg, wherein the one or more prebiotics comprise LH01 Myoviridae, LL5-Siphoviridae, T4D Myoviridae, and LL12-Myoviridae, wherein the inner capsule is disposed within the outer capsule.

In some embodiments, the probiotic blend comprises a total probiotic content of at least about 2 billion CFUs. In some embodiments, the probiotic blend comprises a total probiotic content of about 9 billion CFUs to about 13 billion CFUs. In some embodiments, the probiotic blend comprises a total probiotic content of at least about 11 billion CFUs. In some embodiments, the probiotic blend comprises a total probiotic content of about 11 billion CFUs.

In some embodiments, the outer capsule is a delayed release capsule. In some embodiments, the outer capsule is a delayed release capsule comprising hypromellose and gellan gum. In some embodiments, the inner capsule is an immediate release capsule. In some embodiments, the inner capsule is an immediate release capsule comprising Hypromellose. In some embodiments, the outer capsule and/or the inner capsule further comprise hydroxypropyl methylcellulose, microcrystalline cellulose, magnesium stearate, silicon dioxide, or combinations thereof. In some embodiments, the outer capsule is a delayed release capsule configured to release or substantially release its contents in the intestine following ingestion. In some embodiments, the outer capsule is a delayed release capsule configured to release or substantially release its contents in the intestine following ingestion, and the inner capsule is an immediate release capsule configured to release its contents in the colon following ingestion.

### Properties

In some embodiments, the multi-biotic supplement is opaque. In some embodiments, the multi-biotic supplement is translucent. In some embodiments, the multi-biotic supplement is transparent.

In some embodiments, the multi-biotic supplement is essentially free of ingredients derived from animals. In some embodiments, the multi-biotic supplement may contain less than 1 wt %, less than 0.1 wt %, or less than 0.01 wt % gelatin. In some embodiments, the multi-biotic supplement may be essentially free of gelatin.

In some embodiments of the multi-biotic supplements described herein, refrigeration is not required for the stability of the multi-biotic supplement and/or its components.

### Packaging

### A. Containers and Lids

The multi-biotic supplements provided herein may be packaged in any suitable container. In some embodiments, the container may be a bottle. The container may be of any suitable material. In some embodiments, the container may be plastic. In some embodiments the plastic is about 50%, 60%, 70% 80%, 90%, or 100% Post-Consumer Recycled (PCR). In some embodiments, the container may be glass. In some embodiments, the container includes a lid. In some embodiments, the container includes a removable lid, such as snap-on lid or a screw-top lid. In some embodiments, the lid is fully removable, for example, the lid may be completely separated from the container. In some embodiments, the lid may be connected to the container, for example by a piece of material that is attached to both the cap and the container, such that when the cap is removed from the opening of the container it is still connected to the container.

In some embodiments, the container may be made of one or more plastics. In some embodiments, the container may be essentially free of one or more bisphenol compounds. In some embodiments, the container is essentially free of bisphenol A. In some embodiments, the container may be any suitable color. In some embodiments, the container may be opaque, transparent, or translucent. In some embodiments, the container is opaque. In some embodiments, the lid may be made of one or more plastics. In some embodiments, the lid may be essentially free of one or more bisphenol compounds. In one embodiment, the lid is essentially free of bisphenol A. In some embodiments, the lid is 100% PCR. In some embodiments, the lid may be any suitable color. In some embodiments, the lid may be opaque, transparent, or translucent. In some embodiments, the lid is opaque. In some embodiments, the container and the lid are made from the same material. In some embodiments, the container and the lid are both opaque. In some embodiments, the container and the lid are made from different materials. In some embodiments, the container comprises a bottle and a removable screw-top lid. In some embodiments, the container comprises a bottle and a removable screw-top lid, wherein the bottle and lid are both opaque.

In some embodiments, the container may comprise one or more markings. In some embodiments, the one or more markings are selected from one or more words, symbols, instructions, or slogans. The one or more markings may be any suitable color, or a plurality of colors.

In some embodiments, the container further comprises a desiccant. In some embodiments, the desiccant is disposed within the container. In some embodiments, the desiccant is affixed to an interior surface of the container. In some embodiments, the desiccant is affixed to substantially all interior surfaces of the container for moisture control. In some embodiments, the desiccant is affixed to all interior surfaces of the container. In some embodiments, the desiccant is affixed in a substantially uniform layer on the interior surfaces of the container. In some embodiments, the desiccant is molecular sieves. In some embodiments, the molecular sieves comprise sodium aluminosilicate. In some embodiments, the molecular sieves are 4A molecular sieves.

In some embodiments, the container contains the desiccant in an amount of about 1 g to about 30 g. In some embodiments, the container contains the desiccant in an amount of about 1 g to about 20 g. In some embodiments, the container contains the desiccant in an amount of about 1 g to about 15 g. In some embodiments, the container contains the desiccant in an amount of about 2 g to about 12 g. In some embodiments, the container contains the desiccant in an amount of about 2 g to about 10 g. In some embodiments, the container contains the desiccant in an amount of about 3 g to about 10 g. In some embodiments, the container contains the desiccant in an amount of about 3 g to about 8 g. In some embodiments, the container contains the desiccant in an amount of about 4 g to about 8 g. In some embodiments, the container contains the desiccant in an amount of about 5.5 g.

In some embodiments, the container contains the desiccant in an amount such that a ratio of the volume of the container to the weight of the desiccant ranges from about 5:1 cm³/g to about 50:1 cm³/g. In some embodiments, the container contains the desiccant in an amount such that a ratio of the volume of the container to the weight of the desiccant ranges from about 5:1 cm³/g to about 40:1 cm³/g. In some embodiments, the container contains the desiccant in an amount such that a ratio of the volume of the container to the weight of the desiccant ranges from about 10:1 cm³/g to about 40:1 cm³/g. In some embodiments, the container contains the desiccant in an amount such that a ratio of the volume of the container to the weight of the desiccant ranges from about 15:1 cm³/g to about 30:1 cm³/g. In some embodiments, the container contains the desiccant in an amount such that a ratio of the volume of the container to the weight of the desiccant ranges from about 15:1 cm³/g to about 25:1 cm³/g. In some embodiments, the container contains the desiccant in an amount such that a ratio of the volume of the container to the weight of the desiccant ranges from about 20:1 cm³/g to about 25:1 cm³/g. In some embodiments, the container contains the desiccant in an amount such that a ratio of the volume of the container to the weight of the desiccant is about 22:1 cm³/g. In some embodiments, the container contains the desiccant in an amount such that a ratio of the volume of the container to the weight of the desiccant is about 130:6 cm³/g. In some embodiments, the container contains the desiccant in an amount such that a ratio of the volume of the container to the weight of the desiccant is about 134:6 cm³/g.

### B. Inserts

In some embodiments, the multi-biotic supplement may be packaged with one or more inserts. In some embodiments, the multi-biotic supplement and the one or more inserts may be included in the container described above. In some embodiments, the insert is a scented insert. In some embodiments, the scented insert may be included in the container with the multi-biotic supplement described herein. In some embodiments, the scented insert may associate a desirable odor with the multi-biotic supplement or reduce the perception of an undesirable odor with the multi-biotic supplement, which may increase a subject's compliance with a dosing regimen. For example, the composition may comprise butyric acid, which may impart an undesirable pungent odor. Including a scented insert with a composition that comprises butyric acid may decrease the perception of an undesirable pungent odor, which may in turn increase compliance with the dosing regimen of the dietary supplement composition.

In some embodiments, the scented insert comprises polymer and an odorant agent. In some embodiments, the odorant agent may be derived from natural sources. In some embodiments, the odorant agent comprises a plant oil or one or more compounds derived from plants. In some embodiments, the odorant agent is a plant oil or one or more compounds derived from plants. In some embodiments, the odorant agent is a plant oil. In some embodiments, the plant oil is an oil, extract, or scent of cherry, cinnamon, citrus, clove, ginger, mint, or nutmeg, or any combinations thereof. In some embodiments, the odorant agent comprises a mint oil or mint extract. In some embodiments, the mint oil is peppermint oil. In some embodiments, the odorant agent is derived from Mentha piperita L. In some embodiments, the plant oil is a citrus oil or citrus extract. In some embodiments, the citrus oil or citrus extract may be selected from a grapefruit, lemon, lime, and orange oil or extract. In some embodiments, the scented insert may comprise up to 1%, up to 3%, up to 5%, up to 7%, up to 10%, up to 12%, up to 15%, or up to 20% of the odorant agent by weight. In some embodiments, the scented insert comprises between 1% to 20%, between 5% to 15%, between 8% to 12%, or between 9% to 11% of the odorant agent by weight. In some embodiments, the scented insert comprises about 10% of the odorant agent by weight.

In some embodiments, the polymer is a copolymer. In some embodiments, the copolymer may comprise vinyl acetate. In some embodiments, the copolymer may be a vinyl acetate copolymer. In some embodiments, the polymer may comprise ethylene. In some embodiments, the polymer is ethylene vinyl acetate. The scented insert may comprise up to 99%, up to 97%, up to 95%, up to 92%, up to 90%, up to 88%, up to 85%, or up to 80% polymer by weight. In some embodiments, the scented insert comprises from 99% to 80%, from 95% to 85%, from 92% to 88%, or from 91% to 89% polymer by weight. In some embodiments, the scented insert comprises about 90% polymer by weight. In some embodiments, the scented insert consists essentially of odorant agent and polymer.

In some embodiments, the scented insert may be any suitable shape. In some embodiments, the insert may generally have the shape of a polygon with three, four, five, six, seven, eight, nine, ten, eleven, twelve, thirteen, fourteen, fifteen, or more sides. In some embodiments, the insert may be a triangle; quadrilateral, such as a square, rectangle, rhombus, parallelogram, trapezoid, rhomboid, kite; a pentagon; a hexagon; a heptagon; a octagon; a nonagon; a decagon; a ring; a prism, such as a cube; a sphere; a pyramid, such as a right pyramid, a rectangular pyramid, a rhombic pyramid, or a star pyramid; a cone; a torus; a cylinder; a letter; a symbol; or a word. In some embodiments, the insert may be in the shape of a star, for example a three-pointed star, four-pointed star, five-pointed star, six-pointed star, seven-pointed star, eight-pointed star, nine-pointed star, ten-pointed star, eleven-pointed star, twelve-pointed star, thirteen-pointed star, fourteen-pointed star, fifteen-pointed star, or a star with greater than fifteen points. The star may be symmetrical, or may not have a plane of symmetry. The insert may be generally round, for example be circular or ovoid. In some embodiments, the insert is shaped to emulate another object, for example an animal, plant, or inanimate object. In some embodiments, the insert comprises one or more holes or cutouts.

In some embodiments, the insert may have a generally consistent thickness, for example having a thickness that deviates less than 10%, less than 5%, less than 2%, less than 1%, or less than 0.5%. In some embodiments, the insert comprises at least one point that deviates greater than 15%, greater than 20%, greater than 25%, greater than 30%, or greater than 35% in thickness from at least one other point. In some variations, the insert is relatively flat. In other variations, the insert is generally not flat. In some embodiments, the insert may comprise one or more letters or symbols, for example located on one or more surfaces of the insert. In some embodiments, the one or more letters or symbols may be embossed, punched, or etched into the surface. In some embodiments, the insert is translucent. In some embodiments, the insert is transparent. In some embodiments, the insert is opaque.

### C Additional Packaging

In some embodiments, the container may be further packaged in one or more additional containers. In some embodiments, the additional container is a box. In some embodiments, the box is a cardboard box. In some embodiments, the cardboard box is a corrugated cardboard box. In some embodiments, the box is made of about 50%, 60%, 70%, 80%, 90%, or 100% post consumer waste paper and is fully recyclable. In some embodiments, the multi-biotic supplement is packaged in a container comprising a bottle and a removable screw-top lid, wherein the container is further packaged in an additional container comprising a cardboard box. The additional container may be any suitable color or shape. In some embodiments, the additional container may comprise one or more markings.

In some embodiments, the packaging may further comprise instructions. In some embodiments, the instructions may include one or more pamphlets, sheets, booklets, or other written media. The instructions may be made of any suitable material, such as paper.

### Kits

Provided herein are kits. Generally, kits can contain one or more of the previously described packaging components. In some embodiments, the kit comprises one or more multi-biotic supplements described herein. In some embodiments, the kit comprises one or more containers. In some embodiments the outer container is made of 100% post consumer waste paper and is fully recyclable. In some embodiments, the inner container is a plastic bottle. In some embodiments, the inner container is a plastic bottle (about 50%, 60%, 70%, 80%, 90%, or 100% PCR. In some embodiments, the inner container includes a desiccant affixed to substantially all interior surfaces of the container for moisture control. In some embodiments, the one or more multi-biotic supplements are packaged in the container. The kits described herein may contain instructions. Such kits may also contain one or more scented inserts. In some embodiments, the one or more scented inserts may be disposed within the one or more containers. In some embodiments the inserts comprise about 90% EVA polymer (ethylene vinyl acetate) and 10% of odorant. In some embodiments, the inserts comprise about 80%, about 70%, or about 60% EVA polymer and at least 10%, 20%, or 30% odorant. In some embodiments, the kit comprises one or more multi-biotic supplements, one or more containers, and one or more scented inserts. In some embodiments, the kit comprises one or more multi-biotic supplements, one or more containers, one or more scented inserts, and instructions. In some embodiments, the kit comprises one or more cardboard boxes.

In some embodiments, provided herein is a kit comprising a multi-biotic supplement, as described herein, comprising a postbiotic or postbiotic precursor, a probiotic blend, and a prebiotic blend, and wherein the supplement comprises a first capsule and a second capsule. In certain embodiments, the first capsule comprises the postbiotic or postbiotic precursor, and the second capsule comprises the probiotic blend and the prebiotic blend. In some embodiments, the second capsule is disposed within the first capsule.

### Administration and Use

The multi-biotic supplements described herein may be formulated for administration to a mammalian subject. In some embodiments, the mammalian subject is a human. The composition may be formulated for administration to a woman, a man, or both women and men. The composition may be formulated for administration to a female, a male, or both females and males. In some embodiments, the supplement is administered to a human subject aged 18 or older. In some embodiments, the supplement is administered to a human subject aged 18 or less.

The supplements described herein are intended to be consumed orally. One or more supplements (e.g., capsules) may be administered orally. In some embodiments, the supplements are administered once or more per day. It may be beneficial to administer the supplements described herein only once per day. Thus, in some embodiments, one or more supplements are administered once per day.

In some embodiments, the supplements described herein may be administered with food, prior to consuming food, and/or after consuming food. In some embodiments, it may be beneficial to administer a supplement after a fast. In some embodiments, a fast may comprise 1, 2, 3, 4, 5, 6, 7, 8, 9, 10, 11, or 12 or more hours after consuming food and/or beverage.

Without being held to any theory or mechanism, the supplements described herein may improve and/or support one or more of gut health, digestive health, immune health, and gut barrier function in a subject. In some embodiments, the supplements described herein may provide relief from mild and/or occasional digestive upset and/or diarrhea. In some embodiments, the supplements described herein may support vaginal health. In some embodiments, the supplements described herein may support a subject's microbiome during pregnancy and/or the postpartum period.

The improvement(s) described above may result from the formulation of the supplements described herein. The supplements described herein may increase butyrate concentration in the digestive system. For instance, a supplement may comprise tributyrin. In some embodiments, the tributyrin is metabolized into butyrate in the digestive system. In some embodiments, the tributyrin is metabolized and/or converted into butyrate after bypassing the stomach. For instance, the tributyrin may be metabolized and/or converted into butyrate in the intestines. In some embodiments, the supplements described herein increase the concentration of butyrate in the digestive system of a subject as compared to the concentration prior to administration of the supplement. Cells lining the intestines may utilize butyrate as an energy source.

Additionally or alternatively, the supplements described herein may comprise a prebiotic (e.g., one or more bacteriophage). The prebiotic may selectively stimulate the growth and/or activity of one or a limited number of bacteria in the digestive system, improving the health of a subject. In some embodiments, the prebiotic is one or more bacteriophage that kill and/or decrease the population of unwanted and/or harmful bacteria in the gut. In some embodiments, a prebiotic may increase the growth and/or population of beneficial gut bacteria in a subject relative to the population prior to administration of the supplement.

In some embodiments, two or more of the prebiotics, probiotics, and postbiotics described herein act synergistically to increase butyrate production in the digestive system of a subject. In some embodiments, two or more of the prebiotics, probiotics, and postbiotics described herein act synergistically to increase microbial diversity, increase growth of beneficial bacteria, improve gut barrier function, and/or and help prevent symptoms of leaky gut. In some embodiments, the supplements described herein provide a greater improvement to digestive health of a subject compared to taking two or more of a prebiotic, probiotic, and postbiotic as separate doses and/or administrations.

In some embodiments, the supplements described herein comprise a second capsule disposed in a first capsule. In some embodiments, a supplement comprising a second capsule disposed in a first capsule increases administration compliance in a subject compared to administering two or more capsules separately.

### Enumerated Embodiments

Embodiment I-1. A multi-biotic supplement comprising a postbiotic or postbiotic precursor; a probiotic blend; and a prebiotic blend.
Embodiment I-2. The multi-biotic supplement of embodiment I-1, wherein the supplement comprises a first capsule and a second capsule.
Embodiment I-3. The multi-biotic supplement of embodiment I-2, wherein
   a) the first capsule comprises the postbiotic or postbiotic precursor; and
   b) the second capsule comprises the probiotic blend and the prebiotic blend.
Embodiment I-4. The multi-biotic supplement of embodiment I-2 or I-3, wherein the second capsule is disposed within the first capsule.
Embodiment I-5. The multi-biotic supplement of any one of embodiments I-1 to I-4, wherein the postbiotic or postbiotic precursor is in a solution.
Embodiment I-6. The multi-biotic supplement of embodiment I-5, wherein the solution comprises an oil.
Embodiment I-7. The multi-biotic supplement of embodiment I-6, wherein the oil is a medium-chain triglyceride (MCT) oil.
Embodiment I-8. The multi-biotic supplement of embodiment I-7, wherein the MCT oil is a coconut MCT oil.
Embodiment I-9. The multi-biotic supplement of any one of embodiments I-1 to I-8, wherein the supplement is essentially free of ingredients derived from animals.
Embodiment I-10. The multi-biotic supplement of any one of embodiments I-1 to I-9, wherein the supplement is essentially free of gelatin.
Embodiment I-11. The multi-biotic supplement of any one of embodiments I-1 to I-10, wherein the postbiotic is a postbiotic precursor.
Embodiment I-12. The multi-biotic supplement of any one of embodiments I-1 to I-11, wherein the postbiotic precursor comprises tributyrin.
Embodiment I-13. The multi-biotic supplement of embodiment I-11 or I-12, wherein the postbiotic precursor comprises about 300 mg of tributyrin.
Embodiment I-14. The multi-biotic supplement of any one of embodiments I-11 to I-13, wherein the postbiotic precursor comprises about 350 mg of tributyrin.
Embodiment I-15. The multi-biotic supplement of any one of embodiments I-1 to I-14, wherein the probiotic blend comprises Lactobacillus rhamnosus GG and Bifidobacterium animalis ssp. lactis.
Embodiment I-16. The multi-biotic supplement of any one of embodiments I-1 to I-15, wherein the probiotic blend comprises at least 2 billion CFU of bacteria.
Embodiment I-17. The multi-biotic supplement of any one of embodiments I-1 to I-16, wherein the probiotic blend comprises at least 11 billion CFU of bacteria.
Embodiment I-18. The multi-biotic supplement of any one of embodiments I-1 to I-17, wherein the prebiotic blend comprises LH01-Myoviridae, LL5-Siphoviridae, T4D Myoviridae, and LL12-Myoviridae.
Embodiment I-19. The multi-biotic supplement of any one of embodiments I-1 to I-18, wherein the prebiotic blend comprises about 15 mg of bacteriophage.
Embodiment I-20. The multi-biotic supplement of any one of embodiments I-2 to I-19, wherein the first capsule is a delayed release capsule.
Embodiment I-21. The multi-biotic supplement of embodiment I-20, wherein the delayed release capsule comprises hypromellose and gellan gum.
Embodiment I-22. The multi-biotic supplement of any one of embodiments I-2 to I-21, wherein the second capsule is an immediate release capsule.
Embodiment I-23. The multi-biotic supplement of embodiment I-22, wherein the immediate release capsule comprises hypromellose.
Embodiment I-24. The multi-biotic supplement of any one of embodiments I-1 to I-23, further comprising hydroxypropyl methylcellulose, microcrystalline cellulose, magnesium stearate, silicon dioxide, or combinations thereof.
Embodiment I-25. The multi-biotic supplement of any one of embodiments I-2 to I-24, wherein the first capsule is configured to bypass the stomach of a subject.
Embodiment I-26. The multi-biotic supplement of any one of embodiments I-2 to I-25, wherein the first capsule releases its contents after entering an intestine of a user.
Embodiment I-27. The multi-biotic supplement of any one of embodiments I-2 to I-26, wherein the second capsule is configured to bypass the stomach and small intestine of a subject.
Embodiment I-28. The multi-biotic supplement of any one of embodiments I-2 to I-27, wherein the second capsule survives the small intestine to deliver its contents after entering the colon.
Embodiment I-29. The multi-biotic supplement of any one of embodiments I-2 to I-28, wherein the first capsule comprises tributyrin and the second capsule comprises the probiotic blend and the prebiotic blend; and
   a) the weight ratio of the tributyrin to the prebiotic blend is about 20:1; and
   b) the weight ratio of the probiotic blend to the prebiotic blend is about 8:1.
Embodiment I-30. The multi-biotic supplement of embodiment I-29, wherein the first capsule comprises about 300 mg of tributyrin and wherein the second capsule comprises about 120 mg of the probiotic blend and about 15 mg of the prebiotic blend.
Embodiment I-31. The multi-biotic supplement of embodiment I-30, wherein the 120 mg of the probiotic blend comprises about 11 billion CFU.
Embodiment I-32. A method for improving health in a subject, comprising administering the multi-biotic supplement of any one of embodiments I-1 to I-31 to the subject.
Embodiment I-33. A method for improving and/or supporting one or more of gut health, digestive health, and immune health in a subject, comprising administering the multi-biotic supplement of any one of embodiments I-1 to 1-31.
Embodiment I-34. A kit comprising a bottle with a removable lid, wherein the bottle contains a scented insert and at least one multi-biotic supplement of any one of embodiments I-1 to I-31.
Embodiment I-35. The kit of embodiment I-34, wherein the bottle is glass.
Embodiment I-36. The kit of embodiment I-34, wherein the bottle is plastic.
Embodiment I-37. The kit of any one of embodiments I-34 to I-36, wherein the bottle comprises a desiccant disposed therein.
Embodiment I-38. The kit of any one of embodiments I-34 to I-37, wherein a layer of desiccant is affixed to an interior surface of the bottle.
Embodiment I-39. The kit of embodiment I-37 or I-38, wherein the desiccant comprises molecular sieves.
Embodiment I-40. The kit of embodiment I-37 or I-39, wherein the ratio of the container size (cm³) to desiccant (g) is about 130 to 6.
Embodiment I-41. The kit of any one of embodiments I-34 to I-40, wherein the bottle is opaque.
Embodiment I-42. The kit of any one of embodiments I-34 to I-41, wherein the scented insert comprises one or more markings.
Embodiment I-43. The kit of any one of embodiments I-34 to I-42, wherein the bottle comprises one or more markings.
Embodiment I-44. The kit of embodiment I-42 or I-43, wherein the one or more markings are selected from one or more words, symbols, instructions, or slogans.
Embodiment I-45. The kit of any one of embodiments I-34 to I-44, wherein the scented insert comprises a plant oil or at least one compound derived from plants.
Embodiment I-46. The kit of embodiment I-45, wherein the scented insert comprises peppermint oil.
Embodiment I-47. The kit of embodiment I-45, wherein the scented insert comprises a citrus oil.
Embodiment I-48. The kit of any one of embodiments I-34 to I-47, wherein the bottle is further packaged in one or more additional containers.
Embodiment I-49. The kit of any one of embodiments I-34 to I-48, wherein the bottle is further packaged in a cardboard box.
Embodiment I-50. The kit of any one of embodiments I-34 to I-49, wherein the bottle or the one or more additional containers comprise instructions.

### Examples

The following Examples are merely illustrative and are not meant to limit any aspects of the present disclosure in any way.

### Example 1: Formulation of Exemplary Multi-Biotic Supplement

This example describes the formulation of an exemplary multi-biotic supplement, Supplement A, wherein each capsule contains a postbiotic precursor, a probiotic blend, and a prebiotic blend.

Supplement A was prepared according to the formulation of Table 1. Supplement A was configured with an outer capsule encapsulating a postbiotic and an inner capsule that, in turn, encapsulates a probiotic blend and a prebiotic blend. The outer capsule was a delayed-release Size 00 Natural DRCap® made of hypromellose and gellan gum. Inside the outer capsule was a postbiotic precursor (tributyrin), two excipients (coconut oil in MCT and silicon dioxide), and an inner capsule. The inner capsule was an immediate-release Size 3 Natural VCaps® Plus capsule made of hydroxypropyl cellulose. Within the inner capsule was a probiotic blend containing Lactobacillus rhamnosus GG® (10 billion CFU) and Bifidobacterium animalis ssp. lactis BB-12® (1 billion CFU), a prebiotic blend containing LH01 Myoviridae, LL5-Siphoviridae, T4D Myoviridae, and LL12-Myoviridae, and additives (including hydroxypropyl methylcellulose, microcrystalline cellulose, magnesium stearate, and silicon dioxide).

TABLE 1
Sample multi-biotic supplement formulation (Supplement A).

| Location of | Ingredient | | Amount per |
|---|---|---|---|
| Ingredient | Type | Ingredient | serving |
| Outer | Postbiotic | Tributyrin | 350 mg |
| Capsule | precursor | | |
| | Excipient | Coconut oil in MCT | 85 mg |
| | Excipient | Silicon dioxide | 20 mg |
| | | (Aerosil ® 380) | |
| Inner | Probiotic | Lactobacillus | 120 mg |
| Capsule | blend | rhamnosus GG ® and | |
| | | Bifidobacterium | |
| | | animalis ssp. lactis | |
| | | BB-12 ® | |
| | Prebiotic | LH01-Myoviridae, | 15 mg |
| | blend | LL5-Siphoviridae, | |
| | | T4D-Myoviridae, and | |
| | | LL12-Myoviridae | |
| | Additive | Hydroxypropyl | 48 |
| | | methylcellulose | |
| | Additive | Microcrystalline | 22.5 |
| | | cellulose | |
| | Additive | Magnesium stearate | 1.5 |
| | Additive | Silicon Dioxide | 1 |

### Example 2: Simulation of Supplement Behavior in the Upper Gastrointestinal Tract

The behavior of the supplement of Example 1 in the human upper gastrointestinal tract (GIT) was simulated using a modified Simulation of the Human Intestinal Microbial Ecosystem (SHIME®) model.

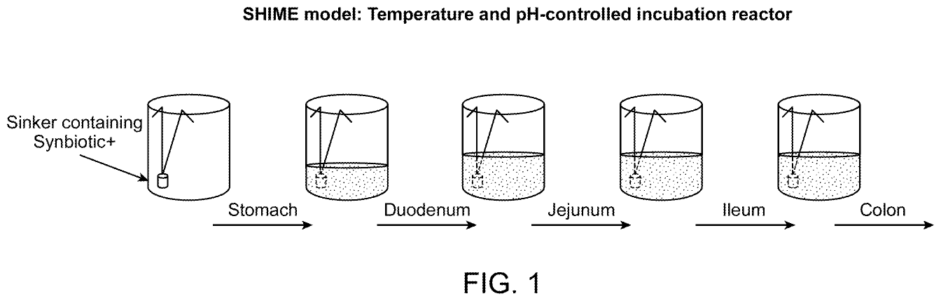

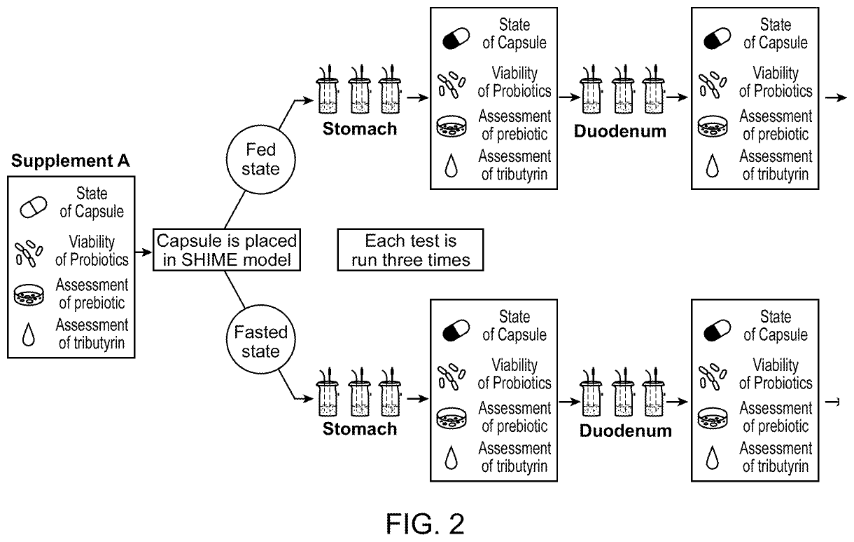

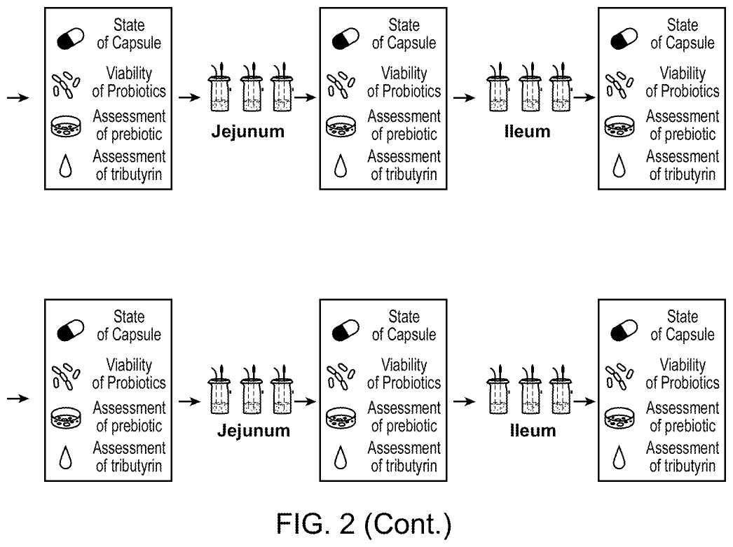

A general schematic of the SHIME model is depicted by FIG. 1. FIG. 2 depicts a schematic representation of the methods used for testing the Supplement A formulation. Supplement A was placed in the SHIME temperature and pH-controlled incubation reactor. Under both fed and fasted states, the supplement was exposed to conditions simulating those of the stomach, the duodenum, the jejunum, and finally the ileum. Under each condition, the state of the capsule, the viability of the probiotics, an assessment of the prebiotics and an assessment of tributyrin were all performed. Each experiment was run three times.

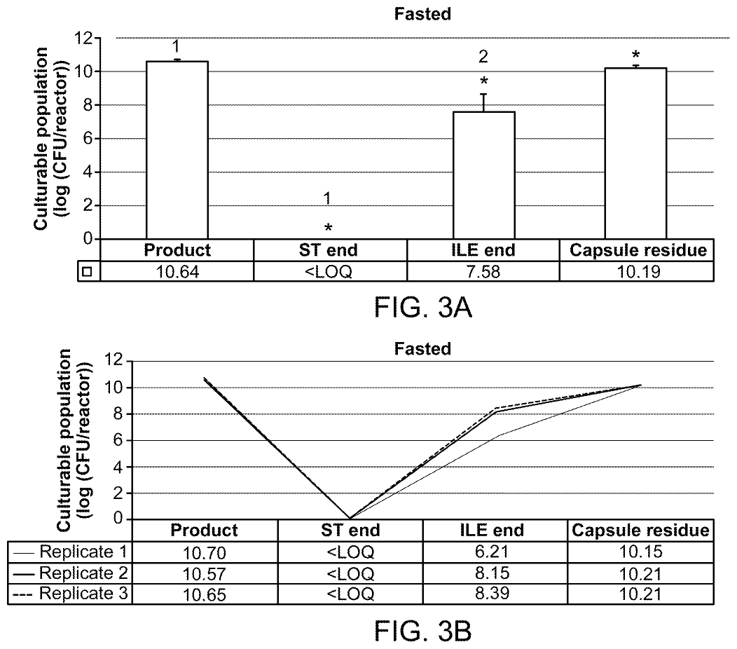

Under fasted conditions, the majority of the probiotics bypassed the stomach and small intestine in a viable form of which more than 11 billion CFU were culturable (FIG. 3A). There were no significant differences between fasted and fed states (data not shown). FIGS. 3A and 3B depict the culturable population densities of probiotics obtained during passage of Supplement A through the upper GIT under fasted conditions (FIG. 3A showing average of three measurements and error bars indicating standard deviation and FIG. 3B showing single replicate measurements; the culturable population density is provided as the logarithm of CFU per reactor). Samples were collected for the product as such and at the end of the stomach (ST end) and ileum (ILE end) phase. Furthermore, the capsule residue was analyzed since full dissolution of the capsules was not obtained by ILE end. For each sampling point, statistical differences from the preceding time point were indicated by means of ‘*’ (p<0.05).

Similarly, the majority of the prebiotic phages survived transit through the stomach and small intestine. Tributyrin was mostly released in the small intestine. The capsule design ensured probiotics and prebiotic phages survived the harsh conditions of the upper gastrointestinal tract for delivery to the colon. The release of tributyrin within the small intestine supported its breakdown by pancreatic enzymes to produce short chain fatty acid butyrate.

### Example 3: Short-Term Colonic Fermentation of the Supplement

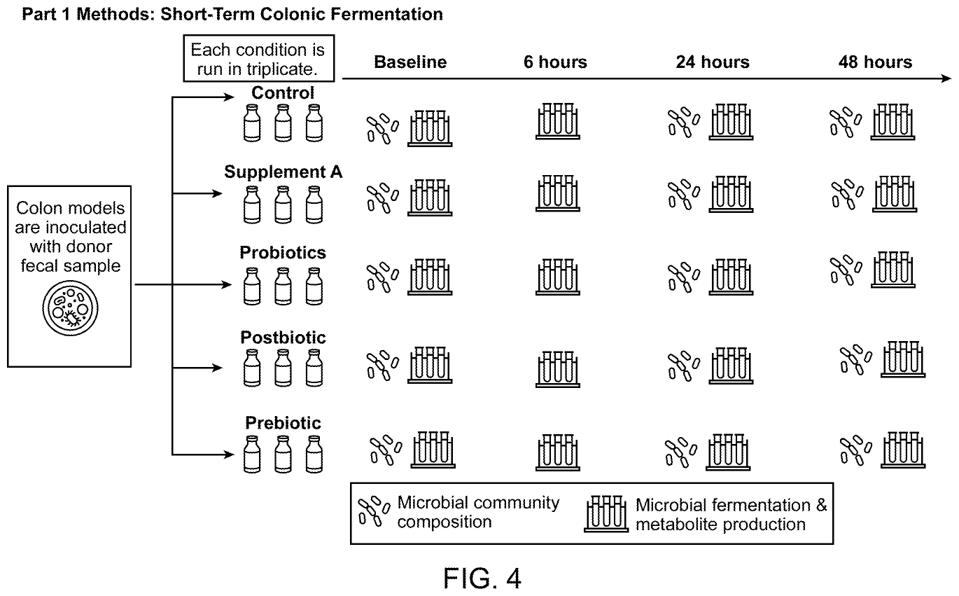

A short-term colonic fermentation was conducted for each condition: Supplement A, postbiotic (“tributyrin”), prebiotic (“PreforPro®”), probiotic blend, and a control). FIG. 4 shows a schematic of the short-term colonic fermentation methods. Each condition was run in triplicate. Product concentrations were selected to reflect an in vivo dose after consuming a single serving of Supplement A. In addition, tributyrin was pre-digested before being added to the reactors. Pre digestion was conducted to reflect the fact that tributyrin released into the small intestine in vivo is partially absorbed after its conversion into butyrate by pancreatic enzymes. Pre-digestion of tributyrin was conducted with simulated passage through the upper gastrointestinal tract, including simulation of oral, gastric, and small intestinal passage with partial absorption via dialysis.

Each test product was added to a reactor with a carbohydrate-supplemented nutritional medium and a freshly prepared fecal sample from a single healthy human donor. The control condition included only the nutritional medium and fecal inoculum and was used to assess baseline fermentation parameters. The reactors were immediately closed with a rubber septum, made anaerobic through flushing with nitrogen, and incubated for 48 hours.

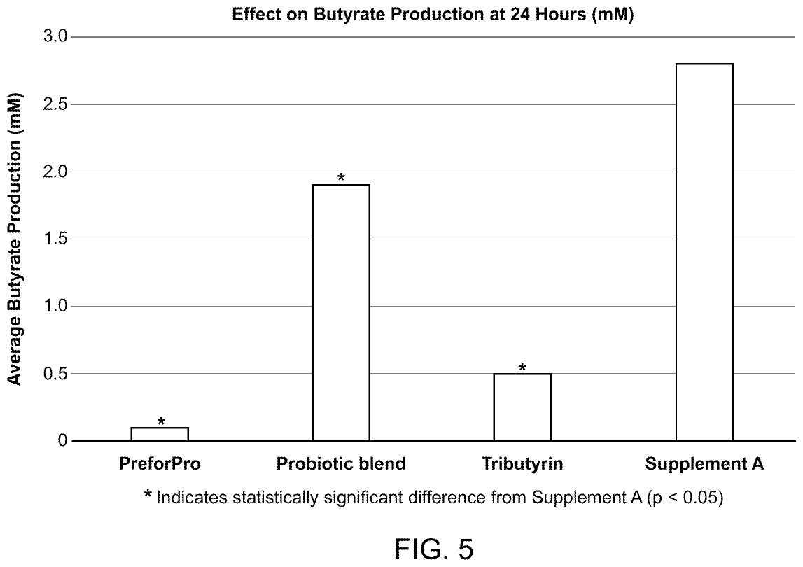

The exemplary supplement of Example 1 stimulated the production of butyrate, lactate, and mild gas. The increased production of lactate in the Supplement A treatment group as well as in the probiotic blend treatment group was likely due to the fermentative activity of the probiotic strains. FIG. 5 shows that the production of butyrate at 24 hours by supplement A was significantly greater compared to each of the individual ingredients: prebiotic blend, probiotic blend, and postbiotic (tributyrin). The effect observed with supplement A was found to be greater than the combined effect of individual ingredients, indicating a synergistic relationship among the prebiotic blend, probiotic blend and tributyrin. The synergistic effect on the production of butyrate can likely be attributed to improved cross-feeding interactions, whereby lactate is provided to butyrate-producing bacteria, as well as the breakdown of tributyrin into butyrate by colonic lipases. The exemplary supplement of Example 1 increased growth of a wide range of bacterial genera at both 24 and 48 hours. The exemplary supplement of Example 1 also significantly increased the alpha-diversity compared with the blank control after 48 hours.

### Example 4: Supplement Effects on the Gut Barrier

This study used an in vitro cell co-culture to create a model of mild intestinal barrier disruption. This method utilizes a co-culture model of intestinal epithelial-like cells (Caco-2 cells) and human monocytes/macrophages (THP1 cells) and is based on the work of Satsu and colleagues (PMID: 17010338) and is described by Daguet et al. (doi: 10.1016r.jff.2015.11.005). Caco-2 cells are allowed to grow into a monolayer that has physiological properties of the intestinal lining, including tight junctions. Tight junction proteins keep adjacent epithelial cells together, which forms a barrier that is virtually impermeable to macromolecules. The “tightness” of tight junctions can be measured as transepithelial electrical resistance (TEER), with a high TEER corresponding to a tighter barrier. THP1 monocytes were originally isolated from a human patient with acute leukemia. When stimulated with phorbol 12-myristate 13-acetate (PMA), these cells develop into macrophage-like cells that are able to adhere and are primed for toll-like receptor (TLR) signaling. When the Caco-2 monolayer is placed on top of PMA-activated THP1 cells, the Caco-2 monolayer becomes disrupted. This creates a model of intestinal barrier disruption, which can be used to test the effects of substances that can protect the gut barrier. TEER was measured after pre-treatment of the cell co-cultures, representing a baseline (0 hour) value. After completion of the 48 hour short-term colonic fermentations, samples from the colonic fermentation suspensions were filtered and sterilized and then added to Caco-2/THP1-Blue cell co-cultures. Application of colonic fermentation samples allows assessment of the effects of each treatment on gut barrier function in a model of mild intestinal barrier disruption. Changes in TEER values were measured after 24 hours.

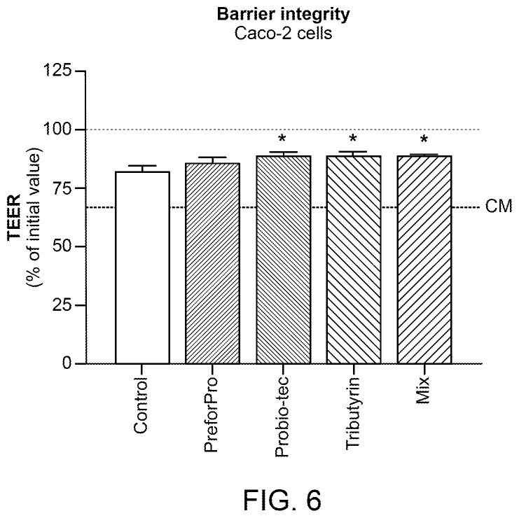

Addition of colonic fermentation samples from Supplement A, probiotics, and tributyrin improved barrier integrity (TEER) compared to the blank control. FIG. 6 shows the effect of colonic batch suspensions on transepithelial electrical resistance (TEER) of the Caco-2/THP1-Blue™ co-cultures. TEER was measured 24 h after pre-treatment of the co-cultures and each 24 h value was normalized to its corresponding Oh value and is shown as percentage of initial value. The grey dotted line represents 100% (initial value). The lower dotted line corresponds to the experimental control CM (complete medium). Data are plotted as mean f SEM. (*) represents statistically significant differences between the control and treatment samples. ($) represents statistically significant differences between the individual compounds: prebiotic (“PreforPro”), probiotic (“Probio-tec”), and postbiotic (“Tributyrin”) and the mix. (*)=p<0.05.

1. A multi-biotic supplement comprising a postbiotic or postbiotic precursor, a probiotic blend; and/or a prebiotic blend.

2. The multi-biotic supplement of claim 1, wherein the supplement comprises a first capsule and a second capsule.

3. The multi-biotic supplement of claim 2, wherein
a) the first capsule comprises the postbiotic or postbiotic precursor; and
b) the second capsule comprises the probiotic blend and the prebiotic blend.

4. The multi-biotic supplement of claim 2, wherein the second capsule is disposed within the first capsule.

5. The multi-biotic supplement of claim 1, wherein the postbiotic or postbiotic precursor is in a solution, and wherein the solution comprises an oil.

6. (canceled)

7. The multi-biotic supplement of claim 5, wherein the oil is a medium-chain triglyceride (MCT) oil, or a coconut MCT oil.

8. (canceled)

9. The multi-biotic supplement of claim 1, wherein the supplement is essentially free of ingredients derived from animals.

10. (canceled)

11. The multi-biotic supplement of claim 1, wherein the postbiotic is a postbiotic precursor.

12. The multi-biotic supplement of claim 1, wherein the postbiotic precursor comprises tributyrin.

13. The multi-biotic supplement of claim 1, wherein the postbiotic precursor comprises about 300 mg of tributyrin, or about 350 mg of tributyrin.

14. (canceled)

15. The multi-biotic supplement of claim 1, wherein the probiotic blend comprises Lactobacillus rhamnosus GG and Bifidobacterium animalis ssp. lactis.

16. The multi-biotic supplement of claim 1, wherein the probiotic blend comprises at least 2 billion CFU of bacteria, or at least 11 billion CFU of bacteria.

17. (canceled)

18. The multi-biotic supplement of claim 1, wherein the prebiotic blend comprises LHO1-Myoviridae, LL5-Siphoviridae, T4D-Myoviridae, and LL12-Myoviridae.

19. The multi-biotic supplement of claim 1, wherein the prebiotic blend comprises about 15 mg of bacteriophage.

20. The multi-biotic supplement of claim 2, wherein the first capsule is a delayed release capsule.

21. The multi-biotic supplement of claim 20, wherein the delayed release capsule comprises hypromellose and gellan gum.

22. The multi-biotic supplement of claim 2, wherein the second capsule is an immediate release capsule.

23. The multi-biotic supplement of claim 22, wherein the immediate release capsule comprises hypromellose.

24. The multi-biotic supplement of 1, further comprising hydroxypropyl methylcellulose, microcrystalline cellulose, magnesium stearate, silicon dioxide, or combinations thereof.

25. The multi-biotic supplement of claim 2, wherein the first capsule is configured to bypass the stomach of a subject, and/or the first capsule releases its contents after entering an intestine of a user.

26. (canceled)

27. The multi-biotic supplement of claim 2, wherein the second capsule is configured to bypass the stomach and small intestine of a subject, and/or the second capsule releases its contents after entering the colon.

28. (canceled)

29. The multi-biotic supplement of claim 2, wherein the first capsule comprises tributyrin and the second capsule comprises the probiotic blend and the prebiotic blend; and
a) the weight ratio of the tributyrin to the prebiotic blend is about 20:1; and
b) the weight ratio of the probiotic blend to the prebiotic blend is about 8:1.

30. The multi-biotic supplement of claim 29, wherein the first capsule comprises about 300 mg of tributyrin and wherein the second capsule comprises about 120 mg of the probiotic blend and about 15 mg of the prebiotic blend.

31. The multi-biotic supplement of claim 30, wherein the 120 mg of the probiotic blend comprises about 11 billion CFU.

32. A method for improving health in a subject, comprising administering the multi-biotic supplement of claim 1 to the subject.

33. A method for improving and/or supporting one or more of gut health, digestive health, and immune health in a subject, comprising administering the multi-biotic supplement of claim 1.

34. A kit comprising a bottle with a removable lid, wherein the bottle contains a scented insert and at least one multi-biotic supplement of claim 1.

35. The kit of claim 34, wherein the bottle is glass or plastic.

36. (canceled)

37. The kit of claim 34, wherein the bottle comprises a desiccant disposed therein, and/or a layer of desiccant is affixed to an interior surface of the bottle.

38. (canceled)

39. The kit of claim 37, wherein the desiccant comprises molecular sieves, and wherein the ratio of the container size (cm³) to desiccant (g) is about 130 to 6.

40. (canceled)

41. The kit of claim 34, wherein (i) the bottle is opaque, (ii) the scented insert comprises one or more markings, and/or (iii) the bottle comprises one or more markings.

42.-44. (canceled)

45. The kit of claim 34, wherein the scented insert comprises a plant oil or at least one compound derived from plants, optionally wherein the scented insert comprises peppermint oil or a citrus oil.

46.-50. (canceled)
